(19) United States
(12) Patent Application Publication
(10) Pub. No.: US 2012/0197533 A1
(43) Pub. Date: Aug. 2, 2012

(54) IDENTIFYING REARRANGEMENTS IN A SEQUENCED GENOME

(75) Inventors: Igor Nazarenko, Sunnyvale, CA (US); Aaron L. Halpern, San Carlos, CA (US); Paolo Carnevali, San Jose, CA (US)

(73) Assignee: Complete Genomics, Inc., Mountain View, CA (US)

(21) Appl. No.: 13/016,824

(22) Filed: Jan. 28, 2011

Related U.S. Application Data
(60) Provisional application No. 61/391,805, filed on Oct. 11, 2010.

(51) Int. Cl. G06F 19/00; G01N 33/48

(52) U.S. Cl. 702/19

(57) ABSTRACT

Methods, apparatuses, and systems for identification of junctions (e.g., resulting from large-scale rearrangements) of a sequenced genome with respect to a human genome reference sequence is provided. For example, false positives can be distinguished from actual junctions. Such false positives can result from many sources, including mismapping, chimeric reactions among the DNA of a sample, and problems with the reference genome. As part of the filtering processes, a base pair resolution (or near base pair resolution) of a junction can be provided. In various implementations, junctions can be identified using discordant mate pairs and/or using a statistical analysis of the length distributions of fragments for local regions of the sample genome. Clinically significant junctions can also be identified so that further analysis can be focused on genomic regions that may have more of an impact on the health of a patient.

### CROSS-REFERENCES TO RELATED APPLICATIONS

The present application claims priority from and is a non-provisional application of U.S. Provisional Application No. 61/391,805, entitled “Nucleic Acid Sequencing and Process” by Nazarenko et al., filed Oct. 11, 2010, the entire contents of which are herein incorporated by reference for all purposes.

This application is also related to commonly owned U.S. patent application Ser. No. 12/770,089 entitled “Method And System For Calling Variations In A Sample Polynucleotide Sequence With Respect To A Reference Polynucleotide Sequence” by Carnevali et al., filed Apr. 29, 2010, the disclosure of which is incorporated by reference in its entirety.

### BACKGROUND

Embodiments of the present invention are related to genomic sequencing, and more particularly to identifying rearrangements in a genome.

Genomic sequencing has progressed in the last few years. Methods can now sequence a sample within a relatively short time period (e.g., days) and with relatively small cost (less than $10,000). One method that provides such speed and efficiency includes the use of paired-end sequencing and a reference genome. A nucleic acid fragment in a sample can have its two ends sequenced with a relatively small number of nucleotides (equivalently base pairs). These mated pairs of sequence reads can then be mapped to one or more reference genomes. The sequences of the mate pair and the expected size of a fragment typically lead the ends of a mate pair to map to locations (defining an interval) that have specific separation, order, and orientation with respect to one another.

However, in some cases, pairs cannot be mapped as expected to a reference genome, and are called discordant pairs. For example, the two ends of a mate pair may each map to the reference but not with the expected order, orientation, and separation, or one end of a mate pair may map to the reference but not the other. This can happen when a rearrangement has occurred in the sample genome relative to the reference genome. Discovering such rearrangements can provide valuable diagnostic and research information. For example, rearrangement typically are the result of disease, such as cancer, or can lead to a greater likelihood of cancer. Besides disease identification, accurate identification of rearrangements can be important for many reasons, such as accurately tracking the heritage of a group of people, as the rearrangement might have occurred several generations previously. But, the determination of when a discordant mate pair results from a rearrangement can be a difficult task, with many false positives appearing.

It is therefore desirable to provide methods, systems, and apparatuses that accurately identify discordant pairs and genomic rearrangements.

### BRIEF SUMMARY

Embodiments of the present invention can provide identification of junctions (e.g., resulting from large-scale rearrangements) of a sequenced genome with respect to a human genome reference sequence. Some embodiments are directed to distinguishing false positives from actual junctions. Such false positives can result from many sources, including mismapping, chimeric reactions among the DNA molecules of a sample, and problems with the reference genome. As part of the filtering processes, a base pair resolution (or near base pair resolution) of a junction can be provided. In various implementations, junctions can be identified using discordant mate pairs and/or using a statistical analysis of the length distributions of fragments for local regions of the sample genome. Certain embodiments are also directed to identifying clinically significant junctions, so that further analysis can be focused on genomic regions that may have more of an impact on the health of a patient.

According to one embodiment, a method is provided for determining whether a junction exists between a sample genome and a reference genome. Results of paired-end sequencing of a plurality of fragments from the biological sample are received. The results include mate pairs of fragments and mappings of the mate pairs to the reference genome. A mate pair includes a first arm read for a first end of a fragment and a corresponding arm read of an opposite end of the fragment. A junction region in the sample genome is identified based on the mappings of the mate pairs to the reference genome. The junction region includes a first edge portion including a first edge of the junction region, a second edge portion including a second (opposite) edge of the junction region, and a potential junction between the first edge and the second edge. A first set of first arm reads is identified, where each at least partially maps to the first edge portion or has a non-negligible probability to at least partially map to the first edge portion based on a mapped location of the respective corresponding arm read. The sequences of the first arm reads of the first set are compared to each other to determine whether a junction exists in the junction region.

According to another embodiment, a method is provided for determining whether a clinically significant junction exists between a sample genome and a reference genome. Results of paired-end sequencing of a plurality of fragments from the biological sample are received. The results include mate pairs of fragments and mappings of the mate pairs to the reference genome. A plurality of discordant mate pairs are determined. A plurality of potential junctions are determined based on the discordant mate pairs. A list of junctions that have appeared in other sample genomes is obtained. For each of the potential junctions, whether the potential junction is on the list is used to determine whether or not the potential junction is a clinically significant junction. In one aspect, a potential junction that is on the list is less likely to be a clinically significant junction.

According to another embodiment, a method is provided for determining whether a junction exists between a sample genome and a reference genome. A plurality of discordant mate pairs is determined based on the mapping results obtained with paired-end sequencing of fragments. The discordant mate pairs are clustered based on locations of the first arms reads and of the corresponding arm reads. For a plurality of the discordant mate pairs of a first cluster, a realignment to the reference genome of each arm of a discordant mate pair is attempted. The realignment of an arm is in a region determined from a length distribution of the fragments. An amount of the plurality of discordant mate pairs of the first cluster that are realigned in a concordant manner is determined. A junction is determined to not exist for the first cluster if the amount is greater than a threshold.

Other embodiments of the invention are directed to systems, computer readable media, and other apparatuses associated with methods described herein.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers can indicate identical or functionally similar elements.

### BRIEF DESCRIPTION OF THE DRAWINGS

FIG. 1 is a flowchart illustrating a method 100 for identifying discordant mate pairs according to embodiments of the present invention.

FIG. 2A shows a diagram of a mapping of a mate pair to a reference genome in a concordant manner according to embodiments of the present invention.

FIG. 2B shows a diagram of a mapping of a mate pair to a reference genome in a discordant manner for types (1) and (2) according to embodiments of the present invention.

FIG. 2C shows a diagram of a mapping of a mate pair to a reference genome in a discordant manner for types (3) and (4) according to embodiments of the present invention.

FIG. 3 is a block diagram of a system according to embodiments of the present invention.

FIG. 4 is a flowchart of a method 400 of analyzing discordant mate pairs to identify potential junctions in a sample genome according to embodiments of the present invention.

FIG. 5 shows a plot 500 of data points for concordant and discordant mate pairs according to embodiments of the present invention.

FIG. 6 shows an example of a region for realignment according to embodiments of the present invention.

FIG. 7 shows a diagram analyzing a junction region to determine whether a junction exists according to embodiments of the present invention.

FIG. 8 is a flowchart of a method 800 for performing junction assembly according to embodiments of the present invention.

FIG. 9 illustrates an example of when the two regions of a sample genome that are connected by a junction are on different chromosomes.

FIG. 10A illustrates a creation of a likely sequence in a junction region based on first arm reads near the junction region and whose corresponding arm reads are in the junction region according to embodiments of the present invention.

FIG. 10B shows a point junction with one boundary and two flank sequences during a calculation according to embodiments of the present invention.

FIGS. 11A-11C shows a discordant mate pair that maps to different regions of the reference genome where repetitive sequences are present according to embodiments of the present invention.

FIG. 12 is a flowchart illustrating a method for identifying discordant pairs that are likely false positives by identifying repetitive elements according to embodiments of the present invention.

FIG. 13 is a flowchart illustrating a method 1300 for identifying common junctions and using the common junctions to filter potential junctions of a sample according to embodiments of the present invention.

FIG. 14 is a flowchart illustrating a method 1400 for determining whether a junction exists between a sample genome and a reference genome using a distribution of fragment lengths according to embodiments of the present invention.

FIG. 15 shows a block diagram of an example computer system 1500 usable with system and methods according to embodiments of the present invention.

### DEFINITIONS

The following definitions may be helpful in providing background for an understanding of embodiments of the invention.

“Polynucleotide”, “nucleic acid”, “oligonucleotide”, “oligo” or grammatical equivalents used herein refers generally to at least two nucleotides covalently linked together in a linear fashion. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones.

The term “reference polynucleotide sequence”, or simply “reference”, refers to a known sequence of nucleotides of a reference organism. The reference may be an entire genome sequence of a reference organism, a portion of a reference genome, a consensus sequence of many reference organisms, a compilation sequence based on different components of different organisms, a collection of genome sequences drawn from a population of organisms, or any other appropriate sequence. The reference may also include information regarding variations of the reference known to be found in a population of organisms. The reference organism may also be specific to the sample being sequenced, possibly a related individual or the same individual, separately drawn (possibly normal to complement cancer sequence).

“Sample polynucleotide sequence”, or simply “sample sequence”, refers to a nucleic acid sequence of a sample or target organism derived from a gene, a regulatory element, genomic DNA, cDNA, RNAs including mRNAs, rRNAs, siRNAs, miRNAs and the like and fragments thereof. A sample polynucleotide sequence may be a nucleic acid from a sample, or a secondary nucleic acid such as a product of an amplification reaction. For a sample polynucleotide sequence or a polynucleotide fragment to be “derived” from a sample polynucleotide (or any polynucleotide) can mean that the sample sequence/polynucleotide fragment is formed by physically, chemically, and/or enzymatically fragmenting a sample polynucleotide (or any other polynucleotide). To be “derived” from a polynucleotide may also mean that the fragment is the result of a replication or amplification of a particular subset of the nucleotide sequence of the source polynucleotide.

As used herein, a “fragment” refers to a nucleic acid molecule that is in a biological sample. Embodiments can perform paired-end sequencing of fragments to obtain a left arm read and a right arm read for each fragment. As used herein, a “mate pair” or “mated reads” refers to the right arm and left are also called a mate pair. A “discordant pair” is when a mate pair does not have the correct orientation or are not within an expected distance in the reference genome. The orientation can be signified with a plus or minus sign for distance.

A “junction” (also called a discontinuity) is the location (a single point or a short region) on the sample genome where the sequences to the left of the junction and to the right of the junction are at different distance, order, or orientation from each other compared to their relationship to one another on a reference genome. This divergence can occur at a single boundary location (e.g., at or between a single base pair) where two distant sequences in the reference genome are joined. The two distant sequences can also be connected (joined) with an intermediate segment between them, and thus there would be two boundaries to the junction at the ends of the intermediate segment. The left and right sequences can be on different chromosomes or on a same chromosome but, for example, 5000 base pairs or more apart on the reference chromosome.

A “junction region” is a region around the junction that defines an area within which a junction has been identified as potentially existing. The edges of a junction region can coincide with the boundaries of a junction, or can be spaced further apart, where an edge region can exist between the boundary of the junction and the edge of the junction region.

A “clinically significant junction” refers to a junction that has been identified as being more likely to cause a new or changed function in a patient, relative to other identified junctions of a group. A “cluster” is used to refer to a group of discordant mate pairs that have similar characteristics, e.g., being associated with a same location in the genome, which may be a junction.

“Mapping” refers to a process which relates an arm read to zero, one or more locations in the reference to which the arm read is similar, e.g., by matching the instantiated arm read to one or more keys within an index corresponding to a location within a reference.

### DETAILED DESCRIPTION

To determine a genome of an organism, fragments from a biological sample can have their two ends sequenced with a relatively small number of nucleotides sequenced at each end. These mated pairs of sequence reads can then be mapped to one or more reference genomes to determine the sample genome. The expected size of a fragment typically leads the ends of a mate pair to map to locations that have specific separation, order, and orientation with respect to one another. However, in some cases, pairs cannot be mapped as expected to a reference genome, and are called discordant pairs. Embodiments can also provide for other ways to obtain discordant mate pairs or partially mapped mate pairs, including: chimeric mate pairs, sequencing errors, mismapping, and situations in which one end of a mate pair maps to the reference but not the other. Discordant mate pairs can occur when a rearrangement, or a large insertion or deletion, has occurred in the sample genome relative to the reference genome.

The accurate identification of rearrangement locations is important as such cases typically are the result of disease, such as cancer, or can lead to a greater likelihood of disease. Such rearrangement can include, for example, a piece of chromosome 2 being at the end of chromosome 4, a section of a chromosome being flipped around so that it has the opposite orientation, or a piece of the genome being deleted. Such rearrangements can cause the loss of a gene, a function of the gene, and a protein created from the gene. Functions can also be lost when parts of the genome need to be near each other to perform the function. For example, for an enhancer that is near an expressed gene, a separation might cause a change in the expression of the gene. Also, since there are changes in which portions of the genome become near each other, a new expression or disregulation can occur. The new function could be that a gene is turned on, which can cause disease. If rearrangement can be identified, then the nature of a disease, such as cancer, might be identified. Thus, the discovery of larger insertions, deletions, duplications, and rearrangements can add value beyond the discovery of small variations. Other advantages can also be realized.

Some embodiments are directed to accurately identifying locations (junctions) of actual rearrangements and other large variations, and distinguishing false positives. One embodiment can provide a junction as a list of putative pairs of loci that are distant in the reference genome, but proximal in the sequenced genome. In various implementations, junctions can be identified using discordant mate pairs and/or using a statistical analysis of the length distributions of fragments for local regions of the sample genome. Certain embodiments are directed to identifying clinically significant junctions so that further analysis can be focused on genomic regions that may have more of an impact on the health of a patient.

### I. Discordant Mate Pairs and Junctions

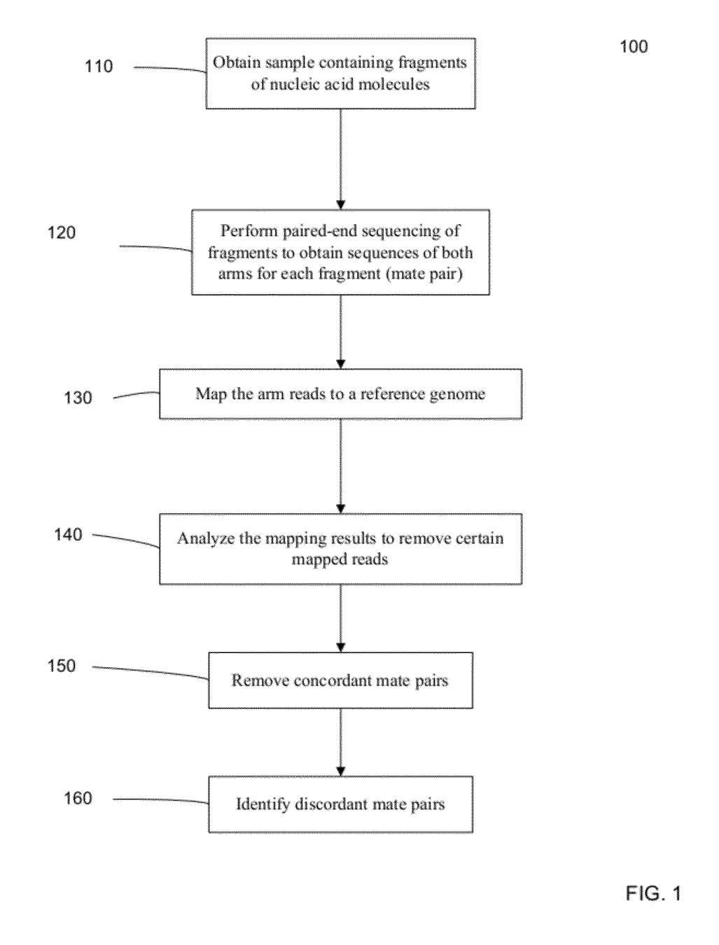

FIG. 1 is a flowchart illustrating a method 100 for identifying discordant mate pairs according to embodiments of the present invention. In one embodiment, the discordant mate pairs can be used to identify potential junctions, which can be analyzed via a variety of embodiments. Method 100, as well as the other methods described herein, can be performed wholly or partially with a computer.

In step 110, a biological sample is obtained from an organism. For example, the organism could be a human, pet, livestock, or other subject in which analysis of the genome is sought. The sample includes fragments of nucleic acid molecules. The fragments can be from any place in the sample genome. The fragments can undergo pre-processing steps, such as amplification, to prepare the sample to obtain better results.

In step 120, a sequencing machine performs paired-end sequencing of fragments from the sample. Each end of a fragment is sequenced (e.g., 20-50 bp). Each sequence of an end of a fragment is called an arm read. The two arm reads are collectively called a mate pair. In one aspect, the two arm reads can be referred to individually as a left arm read or a right arm read. The left and right designation can be relative and depend on an observer's orientation or chosen coordinate system for a reference genome. In another aspect, the two arm reads can be referred to as a first arm read and a corresponding arm read. Such a designation can be more general as it does not depend on a chosen orientation.

In step 130, the arms reads for each mate pair are mapped (aligned) to the reference genome. In one embodiment, any alignment method that permits an independent search for right and left arm locations can be used. In one implementation, the search is guaranteed to find all locations in the genome that match the arm with at most one single-base substitution (mismatches). Another implementation can find some locations that have more mismatches (e.g., up to five mismatches).

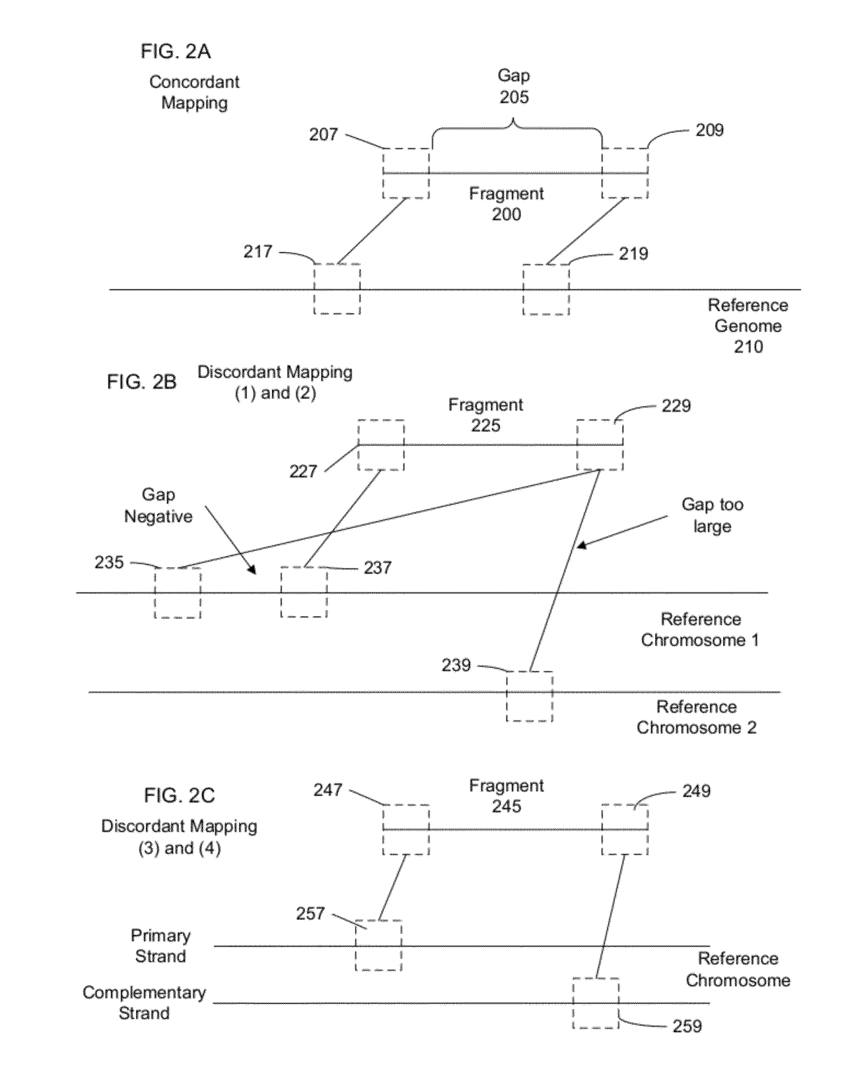

FIG. 2A shows a diagram of a mapping of a mate pair to a reference genome in a concordant manner according to embodiments of the present invention. Fragment 200 has a left arm read 207 and a right arm read 209, which together make up a mate pair. A gap 205 exists on the fragment 200 between the two arm reads of the mate pair.

In the mapping, left arm read 207 maps to a first section 217 of the reference genome 210, and right arm read 209 maps to a second section 219 of the reference genome. The orientation of the arm reads remains the same, and the distance between the arm reads remains approximately the same. Thus, the mapping is concordant. Note that the gap 205 (if it were known) of the fragment does not necessarily match exactly the gap between the first section 217 and the second section 219 after mapping to the reference genome 210.

In step 140, the mapping results can be optionally analyzed to remove certain mapped reads. In one embodiment, all mate pairs that have one and/or both of their arms mapping to more than one location are excluded from further analysis. In another embodiment, mate pairs with a limited number of mapping locations (e.g., less than 3) can be used.

In step 150, mate pairs with at least one concordant mapping are removed from further consideration. Some mate pairs may have a concordant and a discordant mapping. All of the mappings for such a mate pair may be removed. As described above for FIG. 2A, concordant mate pairs are consistent with the reference genome, and thus are not related to a junction. In one embodiment, mate pairs that map on the same strand and chromosome and with a normal mate gap are considered concordant. In one implementation, the normal mate gap range can be defined to cover 99.5% of all orientation- and chromosome-consistent mate pairs.

In step 160, discordant mate pairs are identified. In one aspect, an arm read “points forward” (F) if it is the left mate mapped to primary strand, or a right mate mapped to complementary strand; otherwise we say that the arm read “points backward” (B). Mate-pair orientation can be described by writing the orientation of the mates in the order in which they mapped to the reference. Then normally paired arm reads point toward each other (FB). All abnormal mate pairs can be classified by their orientation into four groups, also called types: (1) oriented FB, but at wrong distance; (2) oriented BF (in other words, out of order); (3) oriented FF (strand mismatched, type I); and (4) oriented BB (strand mismatched, type II). One can verify that a single discontinuity of the sequenced genome with respect to the reference will never generate mates of more than one type; however, some genomic events (e.g., rearrangements, insertions, deletions, etc.) generate multiple discontinuities, e.g., inversion introduces a break at each side of the inverted fragment.

FIG. 2B shows a diagram of a mapping of a mate pair to a reference genome in a discordant manner for types (1) and (2) according to embodiments of the present invention. Fragment 225 has a left arm read 227 and a right arm read 229. To illustrate type (1), left arm read 227 maps to a section 237 of chromosome 1 of the reference genome, and right arm read 229 maps to section 239 of reference chromosome 2 of the reference genome. Thus 237 and 239 are not concordantly mapped. To illustrate type (2), left arm read 227 maps to a section 237 of reference chromosome 1, and right arm read 229 maps to section 235 of reference chromosome 1. As sections 235 and 237 are out of order relative to arm reads 227 and 229, the distance between sections 235 and 237 can be considered negative.

FIG. 2C shows a diagram of a mapping of a mate pair to a reference genome in a discordant manner for types (3) and (4) according to embodiments of the present invention. To illustrate types (3) and (4), left arm read 247 maps to a section 257 of the primary strand of a reference chromosome, and right arm 249 maps to section 259 of the complementary strand of the reference chromosome. Since the orientation of both arm reads is forward (F), there is a mismatch of type (3). If left arm read 247 mapped to the complementary strand, and right arm 249 mapped to the primary strand, then the mismatched orientation would be BB.

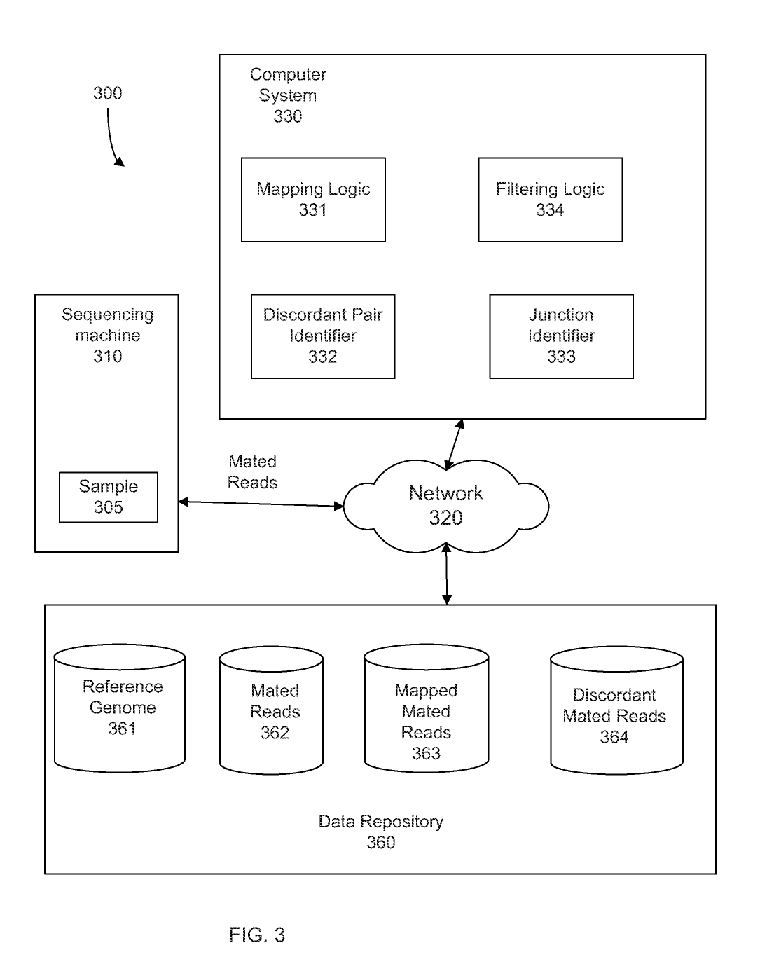

FIG. 3 is a block diagram of a system 300 according to embodiments of the present invention. System 300 can include multiple subsystems, such as sequencing machine 310, computer system 330, and data repository 360. In one embodiment, system 300, or specific subsystems, can be used in any of the methods described herein.

Sequencing machine 310 can receive a biological sample 305 and perform sequencing on fragments in the sample. Any suitable machine that can perform sequencing may be used. The arm reads resulting from the sequencing can be provided as mated pairs to data repository 360, which can store the mated reads 362. Data repository 360 can also store the sequences for the reference genome 361, as well as results of analysis by computer system 330. In various embodiments, data repository 360 can include any one or more of each of the following: hard drives, optical disks, DRAM, flash memory, or any other storage device.

Computer system 330 can be composed of one or more general purpose processors, programmable logic (e.g., a field programmable gate array—FPGA), or application-specific logic (e.g., an application-specific integrated circuit—ASIC), which along with configuration data or software can provide the logic of computer system 330. In one embodiment, computer system 330 has mapping logic 331 for mapping the mated reads 362 to the reference genome 261 to obtain the mapped mated reads 363. A discordant pair identifier 332 can determine discordant mate pairs 364 from the mapped mated reads. A junction identifier 333 can identify potential junctions from the discordant mate pairs 364 or from other characteristics of the mapped mated reads 363. For example, junction identifier 333 can perform clustering of the discordant mate pairs, or perform a statistical analysis of a length distribution of a particular region of the sample genome. Filtering logic 334 can analyze the junctions (potentially including discordant mate pairs, clusters, or other data) to determine whether a potential junction is valid, and/or clinically significant or interesting.

### II. Clustering

As briefly mentioned above, discordant mate pairs may be clustered to determine a potential junction. The clustering may identify junctions that are false positives, and discordant mate pairs that are not the result of actual variations in the sample genome.

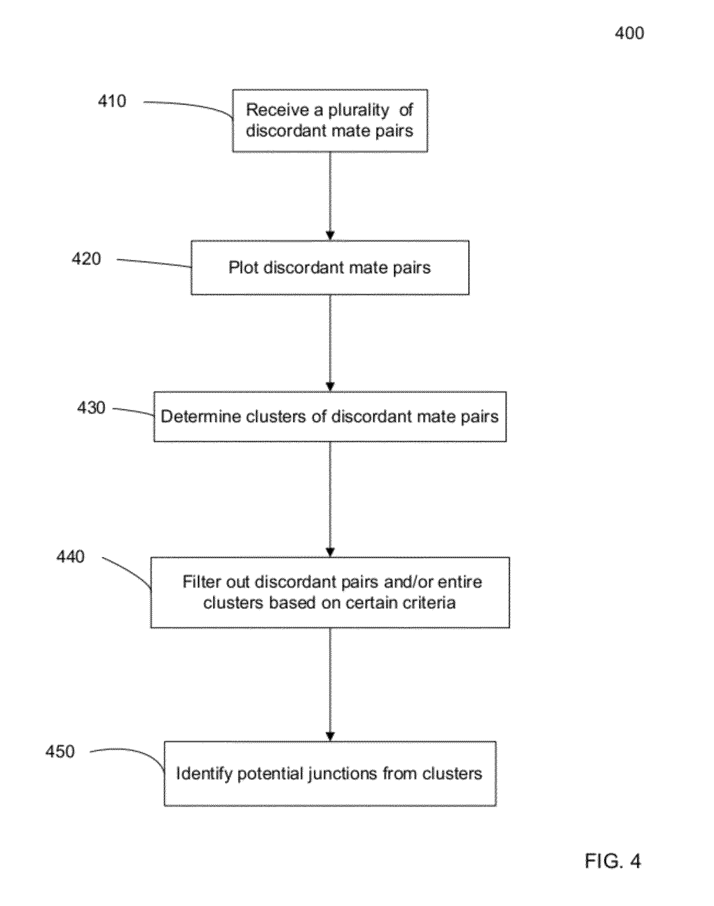

FIG. 4 is a flowchart of a method 400 of analyzing discordant mate pairs to identify potential junctions in a sample genome according to embodiments of the present invention.

In step 410, a plurality of discordant mate pairs are received. In various embodiments, the discordant mate pairs may be determined according to method 100 and variations thereof. In one embodiment, a computer system (e.g., system 330) can receive the discordant mate pairs in a table or list. Each entry for a discordant mate pair can include a location of each arm of the mate pair in the reference genome.

In step 420, the discordant mate pairs are plotted. For example, each discordant mate pair can correspond to a two-dimensional point (the location of each arm read being a different dimension). In one embodiment, the plotting may simply be determining a two-dimensional data point, as a visual plot is not necessary. Based on the two-dimensional data points, distances can be determined between the data points (mate pairs).

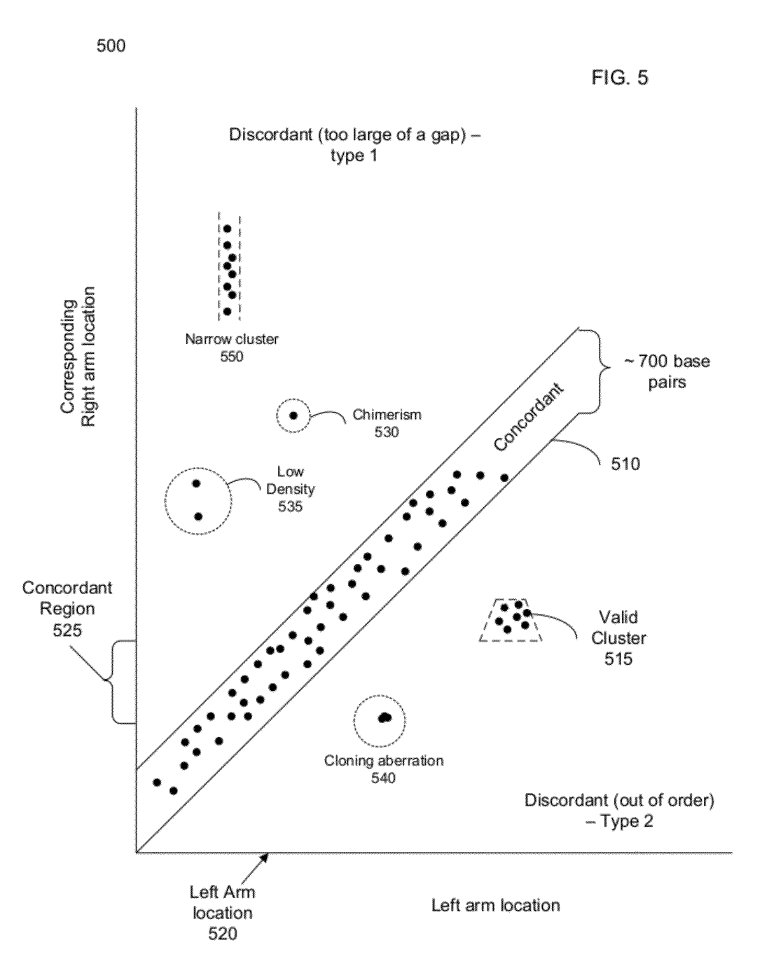

FIG. 5 shows a plot 500 of data points for concordant and discordant mate pairs according to embodiments of the present invention. The X-axis is the location of the left arm, and the Y-axis is the location of the right arm. Thus, each data point denotes a mate pair's position in plot 500. Given a left to right sweep of the genome, the left arm reads begin at zero, and the right arm reads should start at some value greater than zero. Thus, mate pairs that match the reference genome should be above the diagonal starting form zero.

The concordant mate pairs are shown in a diagonal band 510 having one edge being the diagonal starting from zero and the other edge being within a range of expected length of the fragments. Band 510 is labeled as having a height of about 700 base pairs, which is on the high end of the fragment length distribution. A statistical distribution for the length of the sample genome and/or reference genome can be used to determine the height of the band, or effectively what is considered concordant. As an illustration, concordant region 525 corresponds to the left arm location 520.

Data points that are above concordant region 510 are discordant mate pairs of type 1 when the gap distance between the two mapped locations of the arm reads is too large. In this case, a fragment of that size would not be expected, and thus the mate pair would be discordant and might be relate to a junction. As examples, such a junction could result from an insertion or from two distant sections of the genome getting joined.

Data points that are below concordant region 510 are discordant mate pairs of type 2, in that they are out of order. The distance between the left and right arms is negative. In one example, the fragment may be reversed in the sample genome compared to the reference genome. In another example, two distant sections of the genome may have been joined. The data points for the discordant mate pairs of type 3 and 4 can occur anywhere in the plot, including in the concordant region 510. As the mismatch is that the location is on a different strand of a chromosome, the two arm reads could be far apart, close together, or in an opposite order. In one embodiment, discordant mate pairs of different types are clustered separately, with the effect being that the mate pairs of a cluster are of a single type.

In step 430, clusters of discordant mate pairs are determined. A cluster is broadly a group of mate pairs. In plot 500, a cluster of discordant mate pairs are shown as being near each other. In one embodiment, the groups of mate pairs are mutually exclusive, in that a discordant mate pair is within only one cluster, and not part of multiple clusters. In another embodiment, a mate pair can be part of multiple clusters.

In some embodiments, a distance between mate pairs can be used to determine a cluster. In one embodiment, a distance from a mate pair mapped at reference coordinates X1,Y1 (by definition, X1<Y1) to mate pair (X2,Y2) is defined as max(|X2−X1|,|Y2−Y1]). In one implementation, mate pairs can be considered neighbors (in the same cluster) if this distance is below a certain threshold (e.g., 100 bp).

In one embodiment, the clustering can be performed as follows. All discordant mate pairs are designated as “unassigned”. For each unassigned mate pair, its neighbors are found, and the mate pair is then marked as “assigned”. In one implementation, if the mate pair has only one or no neighbors, assume that it is a random chimera and move to the next unassigned mate pair. Otherwise, all neighboring mate pairs can be designated as a part of a new cluster. A cluster can be recursively expanded as follows. For every mate pair in a cluster, its neighboring mate pairs are determined. If the count of mate pairs in the neighborhood is greater than a threshold value (e.g., three), the neighbors are added to the cluster, and the expansion can be repeated. In one aspect, the number of mate pairs used to determine whether to add the mate pairs to the cluster is selected based on a trade-off between false negative and false positive mistakes.

In other embodiments, a more aggressive clustering can be used that allows any compatible mate pairs to be combined into a single cluster. In one aspect, the compatibility can be defined as any two mate pairs that can be caused by the same junction with sufficiently high probability. In one implementation, compatible mate pairs can be data points that are within a same shape, e.g., a trapezoid. In another implementation, compatible mate pairs can be determined as follows. A confidence score for every potential cluster can be computed by determining the ratio of probabilities P(reads|S)/P(reads|R), where P(reads|S) is the probability that all mate pairs in the cluster came from a genome with a discontinuity (the discontinuity chosen to maximize P(reads|S)), and P(reads|R) is the probability that these reads came from the reference genome. In one aspect, this approach takes into account reads with arms that can be aligned to more than one location. In one embodiment, for library of sequencing fragments one can compute a distribution of fragments lengths. Given distance between two data points, one can compute the probability that they are consistent with the same event (that is from same location in a genome, e.g., same discontinuity). In one aspect, the probability can drop to essentially zero past the length of 400 or 500 base pairs, which can be determined from a distribution of the lengths of fragments. For example, a fragment would have a normal length of about 400 or 500 base pairs.

In some embodiments, the discordant mate pairs of each type are clustered separately. For example, only discordant mate pairs of type 1 may be used for one clustering process, with clusters formed only of type 1 clusters. A next clustering process can use only discordant mate pairs of type 2, with clusters only being of type 2, and so on.

In an embodiment where a separate clustering process is initially done for each mate pair type, the resulting clusters within every group can be merged together. For example, two clusters of different types can be merged if no two mate pairs in the merged cluster are more than the normal mate-pair distance apart. Other clustering criteria, e.g., average linkage, can be considered. The “normal mate-pair distance” can be selected based on the known empirically determined distribution of mate-pair gaps in such a way that the normal range covers P=0.995 of all data points with the clusters of discordant mate pairs. In one implementation, P is a tunable parameter, and thus a different value can be selected. The clusters that contained too few mate pairs after the merge can be discarded. Such discarding can also occur for the clustering for a particular type.

In step 440, discordant pairs and/or entire clusters are filtered out based on certain criteria. In one aspect, the filtered discordant mate pairs relate to false positives. In another aspect, the remaining mate pairs are ones that show likely instances of important (e.g., significant) differences from sample genome to reference genome. Filtering out discordant mate pairs encompasses filtering out a cluster, as a cluster is composed of discordant mate pairs. In various embodiments, the criteria used for filtering can be based on a characteristic of a particular mate pair (including characteristics that are mutual to the mate pairs of a cluster) or based on an aggregate property (e.g., a statistical value, such as an average) for the mate pairs of a cluster.

In some embodiments, a criterion is the number of mate pairs in the cluster. For example, a sufficient number of mate pairs must be present in order to continue analysis; otherwise, the cluster might be excluded from further analysis. In one embodiment, if a cluster has only one or two mate pairs, embodiments can assume that it is a random chimera (e.g., chimerism 530), and discard the cluster. A chimerism can be a spurious result of two nucleic acid molecules combining during a biochemical reaction that is part of the preparation of the sample for sequencing. For example, a fragment from one part of the genome can combine with a fragment from another part of the genome. Such combination is not in the sample genome, but occurred during a chemical process for the sequencing. Since it is spurious, there is typically just a low level of occurrences of these chimerisms. Fragments showing real events (e.g., rearrangements in a genome) occur more often as they result from the actual genome, and not just due to a spurious reaction. In another embodiment, one can discard clusters that have less than a specified number of mate pairs, e.g., less than 2 or 3. Even though there are enough neighbors that the data points are probably not the result of a chimerism, the data points are most likely random events, e.g., mismappings.

In other embodiments, a criterion is a density of data points within a cluster. If a cluster corresponds to an actual event in the sample genome, then a sufficient density of data points should appear in the sequencing results. If the cluster does not correspond to a real event, then one would not the data points to be near each other since events would be random. The density for a cluster can be computed and compared to a threshold. Low density cluster 535 is an example of such a cluster. In one embodiment, the density threshold can be derived from the density of data points in the concordant region (e.g., as depicted in FIG. 5). As shown in FIG. 5, the concordant mate pairs are within a band 510, and one data point would typically have another data point within 500 bp. A random event would not typically have a neighbor within about 400 or 500 base pairs on both axes. Thus, a density between data points in concordant region can be compared to a density of data points in a discordant region. For example, if the density is much lower, then one can determine that the cluster is not a real event, e.g., the cluster relates to a chimerism or mismapping. In various embodiments, density can be defined as a number of points per area (which may be obtained using any shape in plot 500, such as a circle, trapezoid, etc), a number of neighbors within a particular distance, and a distance to a neighbor.

In yet other embodiments, too high of a density (e.g., a density value above a threshold) might signal an artificial event. For example, if a chimerism undergoes an amplification (e.g., during a preparation stage of the sequencing), then there may be many data points in the cluster. The cluster would have sufficient density, but the density may be greater than another threshold. Cloning aberration 540 is an example of such a cluster. Each of the data points are on top of each other since each amplified chimeric fragment is identical. Thus, a filter can be if the clustering is too dense. In one embodiment, only unique data points are used in the clustering. Thus, if a second or more data points are the same as an existing data point, then those same data points (e.g., resulting from a cloning aberration) are discarded. In this manner, cloning aberrations can be identified and removed in order to determine a valid cluster of discordant mate pairs from the remaining unique data points. In one implementation, a minimum number of unique data points may be required, regardless of whether copies are discarded.

An embodiment can identify clusters that are narrow in one dimension (e.g., along left or right dimension) and mark them (e.g., for discarding). Such a cluster can be caused by specific mismappings. Such a criterion can be specified based on positional relationships of mate pairs within the cluster. For example, the first and last starting positions of the mappings along one dimension of a cluster must be at least a specified distance (e.g., 50 base pairs) apart from one another.

In one aspect, any filtering criteria can be imposed after each cluster is determined, or after all are determined. In one embodiment, filtering on clusters is performed before additional analysis so that fewer data points need to be analyzed for the additional analysis.

In step 450, potential junctions are identified from clusters. In one embodiment, the remaining junctions after any filtering are identified as potential junctions. As the discordant mate pairs are near each other (e.g., generally within the expected length of a fragment), they are likely associated with the same section of the sample genome (if the mapping is correct), which may be associated with one or more junctions. A potential junction can be defined by a pair of regions of the reference genome that are within a same region in the sample genome. For example, a region can be near the left arm reads of the cluster and a region near the right arm reads of the cluster, both of which may be considered as part of a same region in the sample genome. The clustering can also provide that any two mate pairs that can be caused by the same discontinuity with sufficiently high probability are in a same cluster. In one embodiment, a junction can be limited to mate pairs of only one type.

In one embodiment, a junction can also be determined by clustering arm reads whose corresponding arm reads do not map to the reference genome. Thus, the clustering would be one-dimensional as opposed to two-dimensional. Other methods described herein can be used to identify potential junctions.

Once potential junctions have been identified, the potential junctions can be filtered further, e.g., to discard other false positives or identify junctions of possible clinical importance.

### III. Further Filtering

The potential junctions resulting from the clustering and any filtering on the clusters can be further filtered. In one embodiment, the further filtering can help reduce the false positive rate, to remove discordant mate pairs, clusters, and/or potential junctions that do not reflect actual rearrangements or other discontinuities. As an example, a cluster of false positives can result from two regions of the genome that are similar, which can cause mismappings. Such errors would not be random since mismapping is between only a few regions. In another embodiment, the filtering can identify junctions of more clinical importance than other junctions. In various embodiments, the filters can be performed in any order, and some may not be done. In one embodiment, the filtering order is from least computationally expensive to more expensive.

Various criteria may be used to increase the specificity of the inferred junctions (i.e., reduce false positives). These include, but are not limited to, use of a higher threshold on the number of mate pairs in the cluster defining a junction, success of a junction assembly process, and exclusion of junctions that fall on one side or the other in certain classes of repeats known to be underrepresented in the reference genome, e.g., GAATGn. In the case of identifying a set of putative somatic variations in a tumor sample (e.g., a sample subjected to a mutagenic compound), it may be desirable to further exclude junctions such that one or both ends overlap the ends of junctions in a catalog of junctions derived from an independent collection of ‘normal’ (non-tumor) samples.

A. Realignment

Embodiments can attempt to realign the arm reads of the discordant pairs of a cluster. The realignment can determine whether a concordant mapping is achievable, and that perhaps the initial mapping was in error, e.g., due to overly stringent parameters in the alignment algorithm. In one embodiment, the region where the right arm read should occur with respect to the left arm read (or vice versa) is identified, and the former is subjected to an aggressive match. In one aspect, this can check to make sure that there is not a consistent mismapping of one end of the fragment.

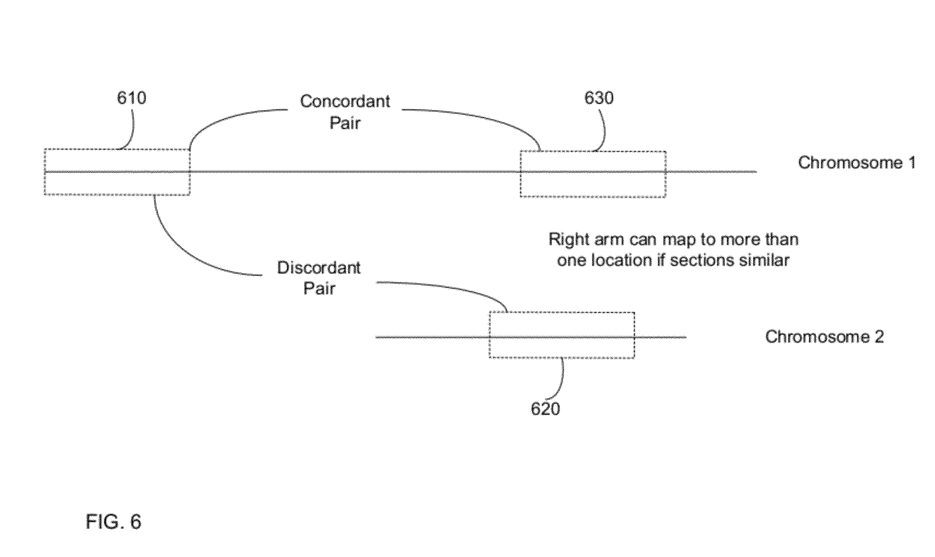

FIG. 6 shows an example of a region for realignment according to embodiments of the present invention. FIG. 6 shows a discordant mate pair of a left arm read 610 and aright arm read 620. As shown, left arm read 610 maps to chromosome 1 of the reference genome, and right arm read 620 maps to chromosome 2 of the reference genome.

In one embodiment, an initial mapping of arm reads to the reference genome can allow only a few errors (e.g., only two errors), and thus only certain possible locations for a mapping may be determined. For example, mappings are allowed to have some skips (indels) or mismatches between the sample genome and the reference genome. However, with different parameters, it may be possible that right arm read 620 does map to a concordant position on chromosome 1, e.g., right arm mapping 630.

In one embodiment, a realignment step can allow for more errors in an attempt to find out if a concordant mapping has a reasonable probability. In another embodiment, since the region for attempted mapping has been localized to the concordant region relative to left arm read 610, more computational effort can be expended to determine the mapping. This aggressive mapping can lead to a mapping that would not have otherwise been detected.

Accordingly, the set of mate pairs can further be filtered by trying to align every read to the reference in the proximity of the other arm read with indels (insertions and deletions) permitted. For example, a group of 10 sequential bases may not map to the reference, but allowing an insertion between the first 5 and second bases might provide alignment. A unit cost may be used for single base mismatches and indels, as well as other scoring schemes being possible with the optimal scheme depending on the sequencing method, known variations in the region, and other criteria as is known to one skilled in the art. A total unit cost may then be used to determine if an alignment exists. If either arm read aligns with relatively small number of edits (e.g., four or less), the mate pair can be discarded. The discarding can reflect that a concordant mapping is more likely than a discordant mapping, or at least that the concordant mapping has a sufficient probability that the mate pair does not reliably reflect a junction.

In one embodiment, a similar realignment can be attempted for left arm 610 onto chromosome 2 at a location concordant with right arm 620. In another embodiment, if either realignment shows a sufficient match, then the mate pair can be discarded from a list (e.g., a cluster) of discordant mate pairs. In one embodiment, a filtering on the number of mate pairs in a cluster can be performed for the first time or again, after a realignment step.

After realignment has been performed, the clusters can be re-evaluated to determine whether a cluster still exists, and thus whether a potential junction still exists. For example, if many of the discordant mate pairs of a cluster are realigned, then the cluster may not satisfy one or more of the above criteria. In another embodiment, the number of successfully realigned clusters can be used to discard all of the mate pairs of a cluster, even though the cluster would survive otherwise. For example, if the number of successfully realigned clusters is greater than a threshold, or if the percentage of successfully realigned clusters is greater than a percentage threshold, then the entire cluster can be discarded or marked.

B. Junction Assembly

Another process that can be considered a filter is junction assembly. The assembly can be performed on any region that is suspected of having a junction to more accurately determine whether a junction does exist. In one example, a sequence that joins two pieces of the reference genome that would otherwise be distant or not connected can be identified, and thus confirm the junction. In one embodiment, the junction assembly, if successful, can provide single-base resolution of the junction, e.g., by reconstructing the sequence of the junction in a local de novo (LDN) manner, as is described in U.S. patent application Ser. No. 12/770,089, which is incorporated by reference. In another embodiment, the junction assembly can be performed on junctions remaining after other filtering steps.

As noted above, a potential junction can be identified as being within a particular region (junction region). Since this region is relatively small, an analysis can be focused. In one embodiment, an arm read that has a non-negligible probability of mapping to the junction region (e.g., the corresponding arm read of the mate pair does map to an area that is approximately a mate pair length away from the junction region) can be used to reconstruct the junction region. The mated reads are subjected to the junction assembly, and any resulting candidate sequence can be subjected to an optimization process, e.g., as described in U.S. patent application Ser. No. 12/770,089.

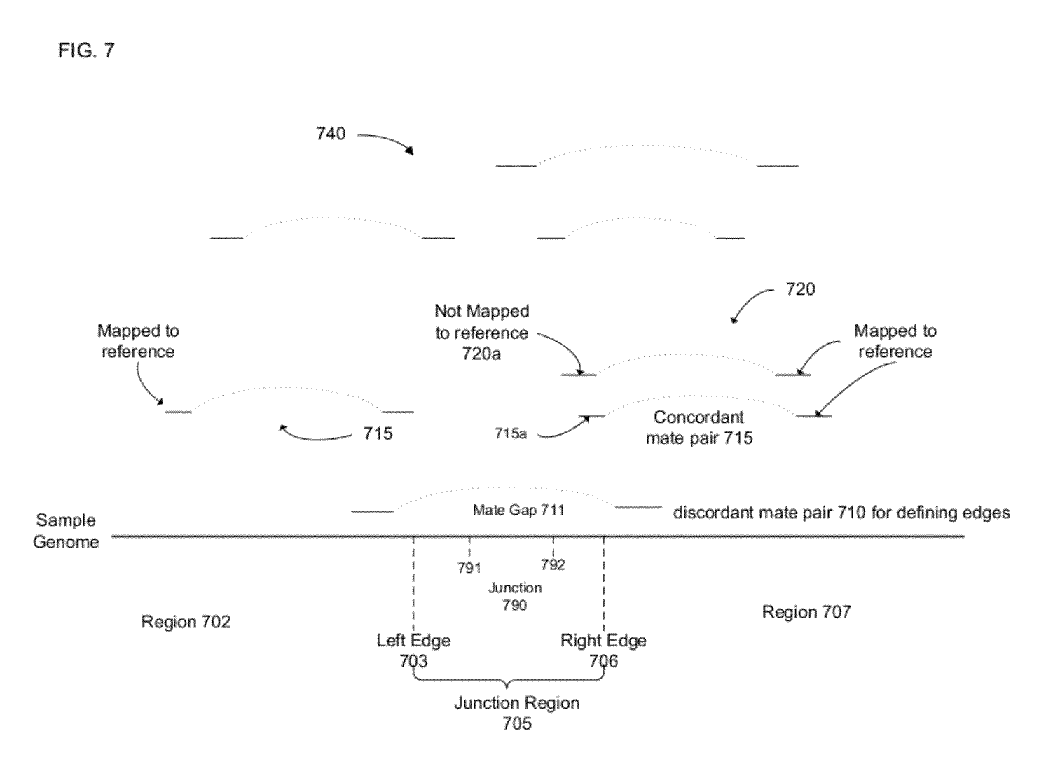

FIG. 7 shows a diagram analyzing a junction region 705 to determine whether a junction (e.g., junction 790) exists according to embodiments of the present invention. The X-axis corresponds to a sequence of the sample genome. There is no Y-axis; the vertical height is simply used to distinguish different mate pairs. The mate pairs are shown with left and right arms and a mate gap (curved dotted line), e.g., mate gap 711.

In the example shown, junction region 705 of the sample genome has been identified as containing a potential junction, as signified by junction 790, which is smaller than the junction region 705. Note that junction 790 is depicted as an example of when a junction actually does exist. A region 702 to the left of junction region 705 (separated by left edge 703) includes arm reads that have initially mapped to a first section of the reference genome. A region 707 to the right of junction region 705 (separated by right edge 706) includes arm reads that have initially mapped to a second section of the reference genome, where the second section is not contiguous with the first section (e.g., to a different chromosome). Thus, a potential junction has been identified in junction region 705.

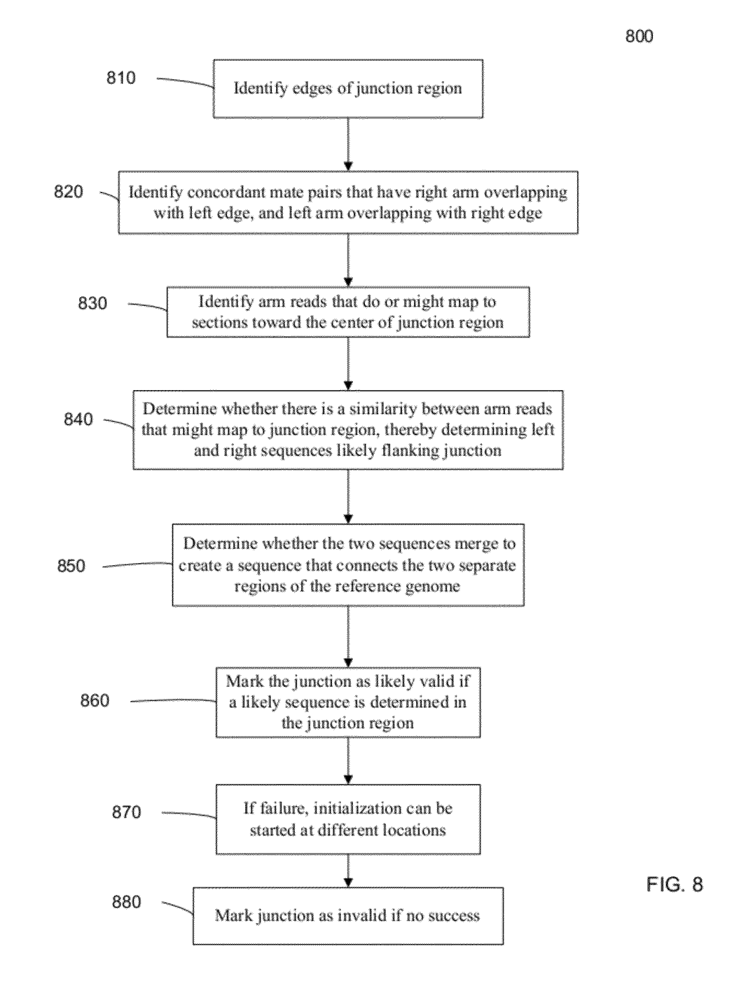

FIG. 8 is a flowchart of a method 800 for performing junction assembly according to embodiments of the present invention. For illustration purposes, method 800 is described with reference to the example in FIG. 7. Method 800 can use the results of other methods, e.g., sequencing and mapping results from a sample genome.

In step 810, edges of a junction region are identified. In one implementation, the junction region can be defined by junction edges, with the junction itself being defined by junction boundaries that are within the edges of the junction region. The junction region can be identified as a region where the sample genome may be different than the reference genome according to various embodiments. In one aspect, the junction region can be selected such that the edges are just beyond where the junction boundaries might be expected.

In one embodiment, discordant mate pairs, such as 710 of FIG. 7, can be used to determine the junction edges. For example, the general area of the junction region can be determined from a cluster of discordant mate pairs, which may all be of the same orientation, as is described above. One or more mate pair 710 can be used to determine the junction edges 703 and 706. These discordant mate pairs have arm reads that are consistent with the respective regions 702 and 707. The arm read(s) furthest to the right for region 702 and furthest to the left for region 707 can define the edges. In one implementation, only the discordant mate pairs of the same cluster are used to define the edges. In some embodiments, an edge is an edge region, which may be estimated from a cluster of discordant mate pairs, or from a statistical analysis of fragment lengths for a region.

In step 820, concordant mate pairs are identified that have right arm overlapping with left edge, and left arm overlapping with right edge. Discordant mate pairs can also be used at an initial stage instead of or in addition to the concordant mate pairs. In one embodiment, the discordant mate pairs may be of a same orientation. In another embodiment, discordant mate pairs having different orientations can be used to analyze a same junction region.

In FIG. 7, concordant mate pairs 715 have arm reads that overlap with the appropriate edges. By using concordant mate pairs, the junction boundaries can be probed further on the two flanks (sides) of the potential junction. Accordingly, the junction can be initialized with sequences from the reference believed to be slightly outside the locations of the junction boundaries 791 and 792; thus, an initial graph (see section V for a description of a graph) can contain two components corresponding to portions of a reference sequence flanking the two boundaries of the junction. In some embodiments, step 820 and the use of concordant mate pairs can be part of step 810 and an identification of the junction edges. In one embodiment, junction edges 703, 706 can correspond to the junction boundaries 791, 792.

In step 830, arm reads that do or might map to sections toward the center of the junction are identified. In this manner, the junction is further probed from the two flanks The contributing mate pairs may be concordant, discordant, or have one mate pair that is not mapped at all. As one moves more toward the center of the junction region, and depending on the size of the junction region, the likelihood of the mate pair being concordant can diminish, and thus other mate pairs may need to be used. In one aspect, for mate pairs where one arm read does not map, the arm read that has been mapped can be identified as a left or right arm read depending on its location relative to the junction region.

In one embodiment, certain left arm reads that map to region 702 and whose corresponding right arms might map to junction region 705 are identified. For example, the corresponding right arms that have a non-negligible likelihood (e.g., greater than 0.5%) of being in junction region 705 can be identified based on the distance of the left arm read from left edge 703 or right edge 706. A distribution of the lengths of the fragments in the sample can be used to determine which range of distances would likely have the corresponding right arm map to junction region 720. In one embodiment, a particular statistical value (e.g., an average) of the length distribution can be used. Other statistical values of a length distribution may be used besides an average. In another embodiment, probabilities of encountering particular lengths may be used. For instance, the length distribution may be peaked (e.g., around 350), with the probability of encountering a length decreasing as the fragment would be too small or too large, and thus an expected range (e.g., 200 to 400 bp) can be determined from when the probabilities start to become too low (e.g., less than 0.5%). Once a range has been determined, left arm reads that are within the distance range away from left edge 703 can be identified, and the corresponding right arms used in the analysis. Corresponding left arm reads can be determined in a similar manner based on right arm mappings to junction region 707.

Mate pair 720 shows an example of where the right arm read maps to the reference genome at a location that is at an expected distance away from where a left arm read might map to junction region 705. However, left arm read 720a of mate pair 720 did not map to the reference genome. This non-mapping may be due to various reasons. For example, left arm 720a may partly map to the first section of the reference genome and partly map to the second section of the reference, and thus the entire sequence was found to not map the reference genome. As another example, if the junction region contains an insertion, left arm 720a may map to the inserted sequence. These two examples may occur if left arm 720a overlaps with a junction boundary. In one embodiment, a junction can have just one boundary, e.g., when two distal points of a genome connect at a single point with no major variations at the point. In another embodiment, the junction can have two boundaries, where the sample genome is different within the boundaries relative to the reference genome. For example, a process that causes two regions of the reference genome to be adjacent to one another in the sample genome can also result in the insertion or modification of additional sequence at the site of the junction.

In step 840, it can be determined whether there exists a similarity among the arm reads that do map to the junction region (e.g., concordant pairs) and the arm reads that might map to the junction region. In other embodiments, only a similarity between arm reads that might map is determined, which as mentioned below can provide an assembly that starts from a reference sequence on one side of the junction and reaches the reference sequence on the other side of the junction. In one embodiment, a similarity can be an exact match in an overlapping region or a match with a small amount of differences. Such a comparison can help determine if the arm read provides new information about the junction region (e.g., if the arm read overlaps an edge, but provides additional sequences toward a center of the junction region). For example, a comparison can determine that left arm read 720a does have a similarity to the left arm read 715a at points where the two arm reads overlap. Such a similarity test can confirm a location of the left arm read 720a, since the exact location is not known a priori. In another embodiment, a similarity can be determined by comparing arm reads to a likely sequence as determined from analyzing the arm reads, or taken from the reference genome outside the junction edge.

In one embodiment, the similarity test can use alignment or optimization processes, e.g., as described in U.S. patent application Ser. No. 12/770,089. Examples of various approaches include overlap-layout-consensus, de Bruijn graph, graph-based, shortest common subsequence, and string graph. A result of the similarity test can be the creation of two sequences that likely occur in the sample genome and flank the junction from the edges toward a center of the junction region. Multiple left and right arm reads that may map to junction region 705 can be determined and used, e.g., arm reads of mate pairs 740.

In step 850, whether the two sequences merge can be determined. In some embodiments, the merging can be confirmed with arm reads that span the two flanking sequences and are similar to the ends of the flanking sequences, thereby completing a sequence from one edge to the other. In one embodiment, if the process results in a graph in which the two initial components are merged into a single component, it is considered to have “succeeded”, and the one or more paths connecting the two components provide hypotheses regarding the sequence of a genomic region wherein two distal regions of the reference genome are proximal in the sample of interest. Such initial hypotheses can be subjected to an optimization process.

In another embodiment, a group of arm reads that do and/or might map to the junction region can be analyzed during a same step to determine whether the arm reads are similar enough to create a sequence that likely spans the junction region. In such an embodiment, flanking sequences may or may not be determined, and the similarities between arm reads can be determined in any order or fashion that can ultimately build a likely sequence in the junction region. In yet another embodiment, if the two sequences do not merge but continue in either direction, then the junction may simply not exist and the junction assembly shows consistency with the reference genome.

In step 860, if a likely sequence is determined in the junction region, the junction is marked as being a likely junction. In one embodiment, the mark can be a binary result of success or not. In another embodiment, the mark can provide a score of the likelihood of the sequence. Either type of mark can be used in a final decision of whether a junction does exist, e.g., in combination with other filters mentioned herein.

In step 870, if method 800 fails to identify a likely sequence, the process can start with an initialization at different locations. For example, the junction edges could be moved farther out or farther in. In one embodiment, if the process does not result in merger of the initial components, the process is repeated using nearby portions of the reference to initialize the graph. Such a process can provide robustness to errors in the mate pair-derived estimates of the junction edges as well as small sequence variants near the junction boundaries. For example, locations −30, −20, −10, +10, +20, +30 bases from each of the initial edges may be tried. If after trying such alternative locations no success is achieved, the junction assembly can be considered to have “failed”.

In step 880, if the junction assembly fails, the potential junction can be marked as likely not valid, e.g., a false positive. In one embodiment, the junction can be removed from a list of potential junctions. For example, a cluster associated with the junction can be discarded. In another embodiment, the mark can be used as a factor for determining whether a junction is likely to exist or does exist.

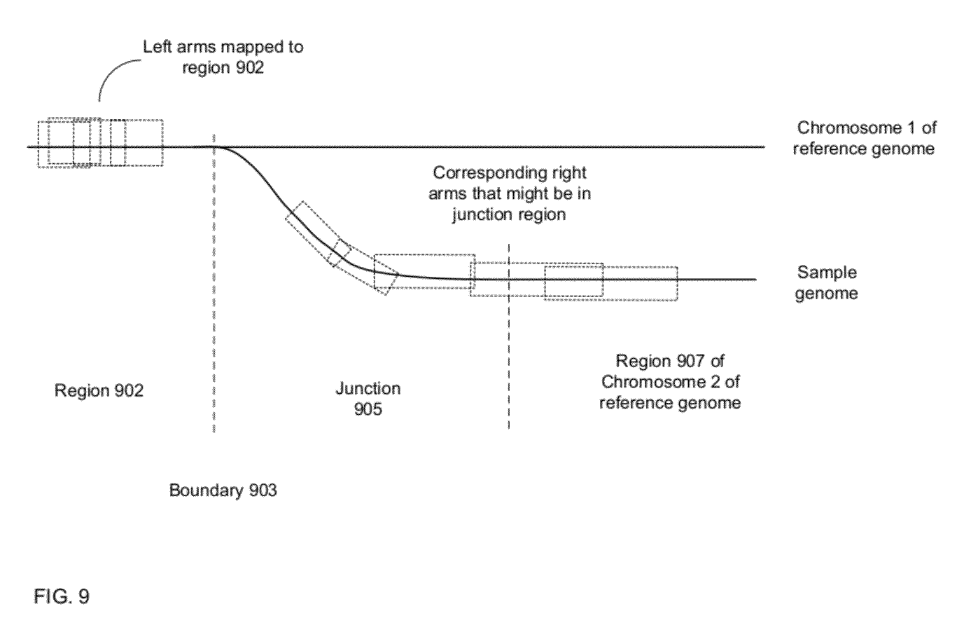

FIG. 9 illustrates an example of when the two regions of a sample genome that are connected by a junction are on different chromosomes. Chromosome 1 of the reference genome is shown as a horizontal line. The sample genome is shown as the line that starts off consistent with chromosome 1, but begins to diverge at the left boundary 903 of the junction 905. Therefore, junction 905 connects region 902 of chromosome 1 to region 907 of chromosome 2. As shown, edges of a junction region around junction 905 can be considered to correspond to the junction boundaries, or the edges are just not shown for ease of presentation.

The left arms reads that map to region 902 are shown as boxes with dotted lines. The left arms can overlap with each other, as is shown. The right arm reads that correspond to the left arm reads are shown in junction 905 and in region 907. As described above, these right arm reads can be used to trace back from reference genome 2 until a boundary is found, and then can also be used to determine the sequence in the boundary.

The right arm reads are shown progressing from region 907 up the sample genome toward a connection with chromosome 1. The right arm reads can be assembled to provide the likely sequence in the junction. Left arm reads that might be in junction 905 can also be used, as is described herein. A junction can also be a single point so that the connection between regions 902 and 907 would be more abrupt (e.g., like a step function). Although method 800 mentions initially using right arm reads that overlap a left edge or boundary, left or right arm reads can be used for any part of a junction or junction region.

In one embodiment, the determination of the sequence in the junction is a de novo calculation since the reference genome does not contribute to the junction, but is simply used to determine the boundaries with the actual sequenced fragments being used to determine the sequence. In one aspect, this de novo calculation can be effective since only a small part of the genome is being analyzed and only a small fraction of the full mate pair data set can be included.

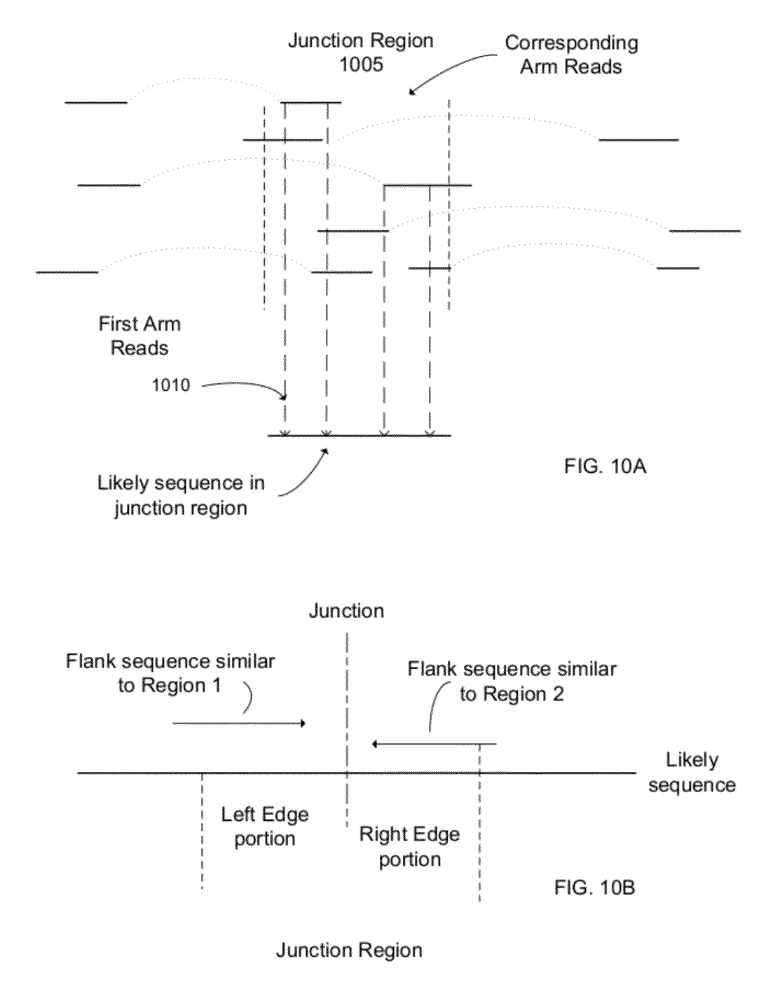

FIG. 10A illustrates a creation of a likely sequence in a junction region based on first arm reads near the junction region and whose corresponding arm reads are in the junction region according to embodiments of the present invention. The mate pairs are shown with first arm reads outside of the junction region 1005 and corresponding arm reads that are in junction region 1005. Note that the use of first and corresponding arm reads reflects the relationship of the mate pair to the region of interest, such that either arm of a mate pair may act as the first or corresponding arm read. The corresponding arm reads are shown being combined to create a contiguous sequence that is the likely sequence in junction region 1005. For ease of presentation, only a few corresponding arm reads are shown, whereas many arm reads may be used.

Arrows 1010 show places where at least some of the corresponding arm reads overlap, and thus where the locations and base pairs of the arm reads can be compared to determine the likely sequence. In one embodiment, if the corresponding arm reads are sufficiently consistent (e.g., matching with an accuracy above a threshold), a likely sequence can be determined. Depending on the consistency of matching, the likely sequence can have a varying likelihood of being accurate. In one embodiment, knowing the likely sequence within an accuracy of a base can distinguish very different functional consequences; for instance, it may reveal whether two genes are brought together “in frame” (sensible protein product may result).

FIG. 10B shows a junction and two flank sequences during a calculation according to embodiments of the present invention. The junction is shown in the middle of a junction region, with left and right edge portions on either side of junction. The two flank sequences match the reference chromosome at different regions (regions 1 and 2). The two flank sequences can be calculated from embodiments of method 800. The arrows signify that sequences are being assembled from the edges of junction region toward the junction.

In one embodiment, the junction area (sequences within boundaries of junction) can be crept up from the left and right so that the final junction area is small (e.g., around 10 bp). This finite size for the junction can result since not all of the genome is actually sequenced, and thus some area of unknown sequence would be expected. In one case, a single boundary can result if one or more arm reads straddle the junction. In one embodiment, regardless of exact size of junction, the size can be reduced enough (and information from the junction region obtained) that important functional aspects and consequences of the junction can be determined.

The junction assembly can fail in a variety of ways. One way is if no (or just few) corresponding arms are found to be in the junction region, and thus the initial mate pairs that suggest the connection of region 1 to region 2 may not be correct data points. In various embodiments, whether or not the junction assembly process succeeds or fails can be dependent on an accuracy of the sequence determined, the number of arm reads in the junction region, the size of the junction region, etc. Also, if no transition is found, e.g., the likely sequence is consistent with the reference genome then data points (discordant mate pairs) suggesting a discordance may be wrong. In another embodiment, how well the junction region is determined can provide a confidence score that may be used for ranking or filtering in later analyses.

C. Removing Repeat Sequences

In one embodiment, a filter on the potential junctions uses repeat sequences (also called repetitive elements) to identify possible false positives. Repeat sequences are consensus sequences that appear often in a genome, e.g., the reference genome. Repeat sequences can result from ancient viruses that are now part of the genome, and can be disruptive if located in middle of a gene. Short interspersed nuclear elements (e.g., Alu) and long interspersed nuclear elements are examples of common sequences that occur throughout the human genome.

Certain repeats may be known to not be properly represented in the human genome. Also, there may be so many of the repeat sequences that exactly where they are located is not known. These problems with the repeat elements can lead to artifacts in the mapping. For example, a sequence A might be related to a sequence B (which can be a repetitive element) in a first chromosome, but B might also appear in another chromosome. The mapping algorithm might make an error and connect A in the first chromosome to the B in the other chromosome, e.g., when A is in a first arm read and B is in the corresponding arm read.

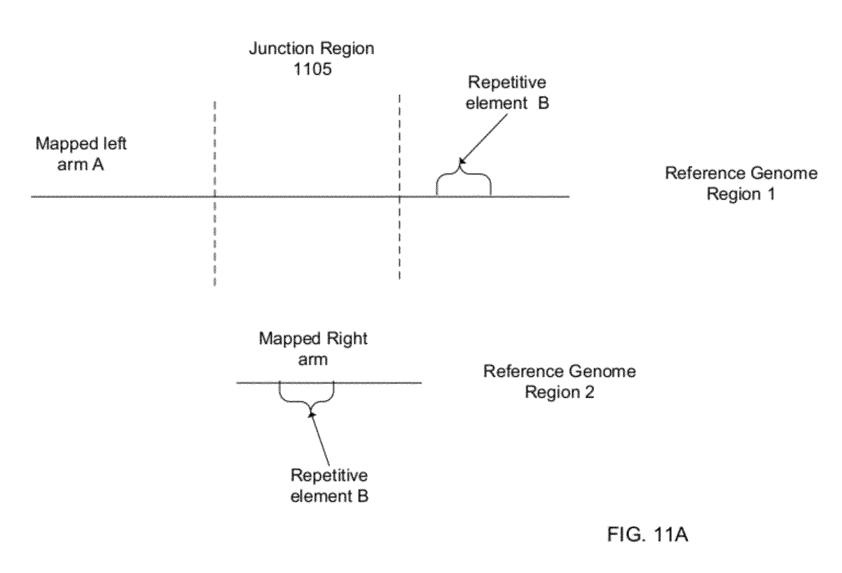

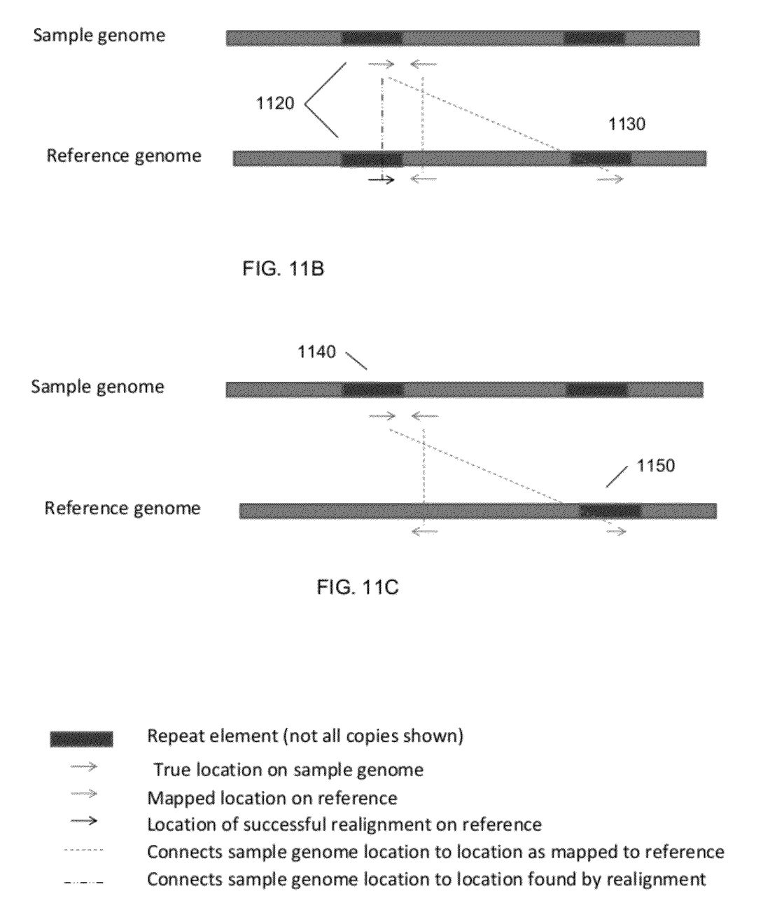

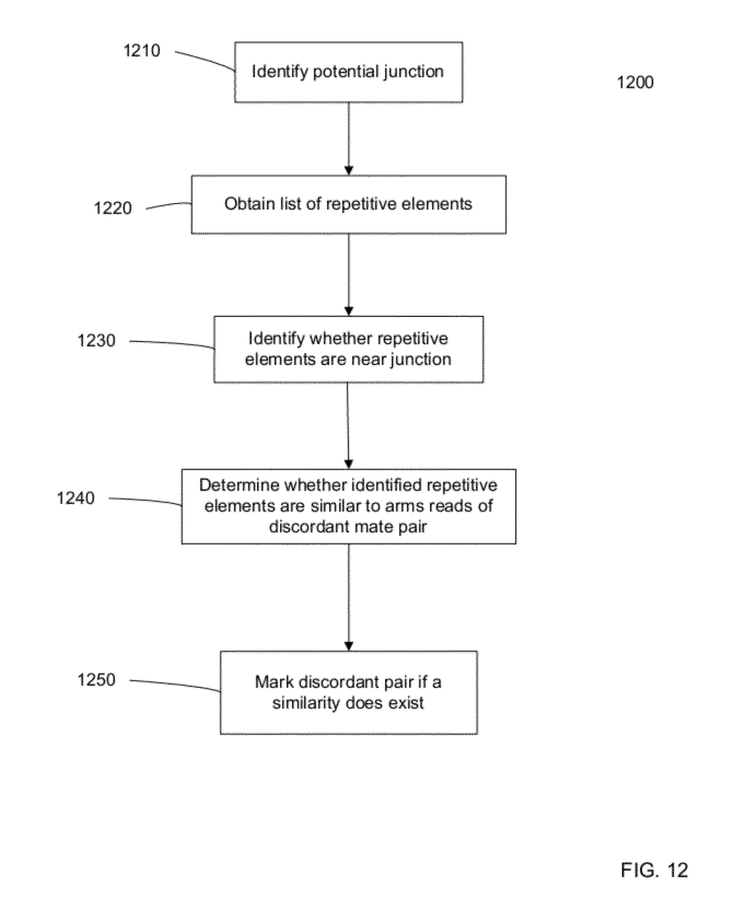

FIG. 11A shows a discordant mate pair that maps to different regions of the reference genome where repetitive sequences are present. Region 1 (e.g., a first chromosome) of the reference genome can be a site of an errant discordant mate pair, resulting in an errant identification of a potential junction. The left arm A of the discordant pair maps to the left side of the junction region 1105. The right arm of the discordant pair maps to a region 2 (e.g., a second chromosome) of the reference genome, specifically to a repetitive element B in region 2. Thus, a junction region 1105 is identified.

However, the repetitive element B also exists to the right of the junction region. Accordingly, the mapping maybe in error, since the right arm could have been mapped to a concordant location on region 1. Thus, there is a high likelihood of the right arm being mismapped to the region 2, even though it may more properly maps to region 1. Thus, a junction does not truly exist since the right arm actually derives from region 1. By identifying one side of a junction as involving a repeat element of a certain class, such junctions can be identified as likely false positives. In one embodiment, the false positive could be identified in a realignment step, e.g., where alignment is attempted for region 1.

What constitutes a repetitive sequence can be defined by a list of sequences in a database. These repetitive sequences in the list can be compared to the reference genome in or near the possible junction region. In one embodiment, the list of particular repetitive elements can be identified as those that are not properly represented in the reference genome. An example of not being properly represented, might be when actual repetitive sequence appears to be unique in the reference genome. Thus, when mapping, the other possible mappings will not be identified. One embodiment uses certain classes of repeats to identify problematic discordant pairs that might have mismapping problems. Example classes of repeats include: ALR/Alpha, (GAATG)n, HSAT4, HSATII, LSU-rRNA_Hsa, and SSU-rRNA_Hsa.

FIG. 12 is a flowchart illustrating a method for identifying discordant pairs that are likely false positives by identifying repetitive elements according to embodiments of the present invention. In step 1210, a potential junction is identified. The junction can be determined by any embodiments described herein. The junction can be identified as being within a junction region defined by edges.

In step 1220, a list of repetitive elements is obtained. In various embodiments, the list is obtained from a database, RAM, or cache, or other suitable memory. The repetitive elements on the list may be associated with problems in mapping and/or identifying junctions. In one embodiment, a user can select which repetitive elements to use, which may occur after a suggested list is provided. In another embodiment, the list is set to a default and cannot be changed.

In step 1230, whether repetitive elements are near the potential junction is determined. In one implementation, each repetitive element in the list can be searched for near the junction. In some embodiments, a repetitive element can be considered near when it is within a specified number of base pairs of the junction. In one embodiment, step 1230 can be performed after a junction region has been identified, and the edges of a junction region can be used to determine whether a repetitive element is present. In one example, the junction region can be identified simply from a cluster of discordant pairs (a relatively large region) or from junction assembly, thereby providing a relatively small junction region. The reference genome can be analyzed in the junction region. If the junction region or an adjacent region (e.g., as defined by a distance cutoff) contains repeat sequences from the list, then the likelihood of a mismapping can be large. In one embodiment, an overlap between a footprint (coverage) of the mappings of the mate pairs and a repeat annotated area is required to provide an affirmative response to step 1230.

In the example of FIG. 11A, the repetitive element B would be identified as being near the junction region 1105. In one embodiment, if an identified element is found, then the junction can be marked as likely a false positive, which can lead to discarding as a false positive.

In step 1240, whether the identified repetitive elements are similar to arms reads of discordant mate pairs is determined. This step can be done as a check to see if the fact that a repetitive element is near the junction is a likely cause of the determination that the mate pair is discordant. For example, in FIG. 11A, a check can be made to determine whether the repetitive element B is similar to the right arm read. Since the mapped right arm does contain the repetitive element B (or vice versa), there is a likelihood of a mismapping. However, if the identified repetitive element is not similar to the corresponding arm read, then a mismapping may not be likely. The similarity can be determined by knowing where the repetitive element can be found in the reference genome, and determining whether the discordant mate pairs have been mapped to that region.

In step 1250, if a similarity does exist, then the discordant mate pair can be marked. Marked discordant mate pairs can be discarded based on this factor or in conjunction with other results. In one embodiment, the discordant pair can be marked as involving a repetitive element. Other processes can then use this information to determine whether further analysis is performed or whether the pair should be discarded (possibly using other information from other analyses). How similar an arm read is to a repetitive element, or how close a part of the reference genome is to a repetitive element can be factors in determining a confidence score of whether a discordant pair should be rejected.

In one embodiment, if other discordant mate pairs do exist and are not similar to the identified repetitive element, then it may be determined to not mark or discard the junction as a false positive. However, if all, a majority, or a certain threshold amount of the discordant mate pairs of a cluster associated with the injunction have an arm read that contains an repetitive element that was identified near the junction, then the junction can be marked or discarded. In yet another embodiment, a determination of whether an arm read contains a repetitive element (and possibly if the arm read is long enough) can be used, without the need of identifying if the repetitive element is near the junction.

FIG. 11B illustrates another discordant mate pair that is mismapped to a region of the reference genome where repetitive sequences are present. As shown, the sample genome and reference genome both have the copy of the repeat 1120 that is the true source of the left arm read of the mate pair, as well as additional copies. In this example, due perhaps to a small sequence variant between the sample and reference genomes in the source copy, there is a mismapping to repeat 1130 (and the correct mapping is not found). However, during a realignment phase, the correct location can be confirmed. Accordingly, in one aspect, no cluster is generated.

FIG. 11C illustrates another discordant mate pair resulting from an insertion of a repeat. Sample genome has an additional copy 1140 of the repeat element. When a mate pair with one arm in the extra copy and one arm outside it is mapped onto the reference genome, the arm from inside the repeat can be mapped to an instance 1150 of the repeat that is present in the reference genome. In this example, attempted realignment to the reference will not find any way to produce a concordant mapping of the pair. If this happens with several mate pairs, it can generate a cluster associated with a junction. In one embodiment, the cluster can be analyzed with junction assembly and the repeat can be identified. The fact that one arm read corresponds to a region of the reference containing one of the designated repeat classes can cause the junction to be marked or filtered. The marking can allow for further analysis to confirm whether a junction does or does not exist, while filtering may provide a quicker decision; and an instance of an insertion of a repeat may not be clinically significant.

E. Baselining

In one embodiment, a filter can check for junctions that are either common errors (false positives) or valid junctions that are common enough that the junction is not likely to be clinically significant. Common false positives can reoccur among different samples, e.g., when certain inaccuracies exist in the reference genome or in the case of common mismappings. The latter may be actual junctions that occur in a certain segment of the population among healthy individuals, and thus are not generally of clinical importance (e.g., related to a rare disease). One embodiment does not distinguish between the two types of junctions, which can be acceptable if clinically non-significant junctions are not desired.

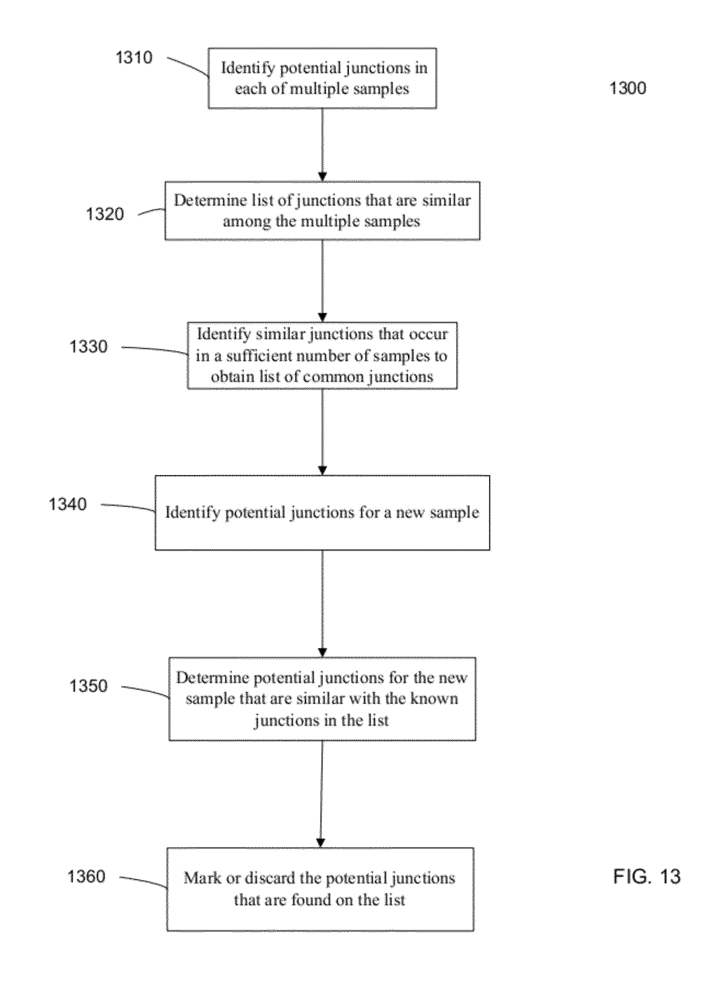

FIG. 13 is a flowchart illustrating a method 1300 for identifying common junctions and using the common junctions to filter potential junctions of a sample according to embodiments of the present invention. Method 1300 can be performed in combination with any of the other filters, or done independently. In one embodiment, the initial steps of identifying common junctions can occur well before any analysis of a current sample.

In step 1310, potential junctions are identified in each of multiple samples. The potential junction can be identified using any of the embodiments mentioned herein. Any number of other filters can also be used to discriminate false positives. In one embodiment, the potential junctions are identified using the same method for each sample.

In one embodiment, a junction could be defined as the connection of two sections of a reference. Thus, a list item could have two entries for the two sections of the reference genome that are connected by the junction. In another embodiment, a junction is stored as the edges of a junction region or actual boundaries of the junction. In one implementation, an orientation of the junction can be stored, where the orientation corresponds to the type of discordant pairs that are used to identify the junction. Embodiments for determining the edges or boundaries are described herein, such as using the closest (innermost) locations of arm reads for a cluster of discordant mate pairs, boundaries from junction assembly, and a use of length distributions of a particular region.

In step 1320, a list of junctions that are similar among the multiple samples is determined. In one aspect, this list can be used as an approximation to junctions that appear often in a population. In one embodiment, the list can be obtained once and thus be static. In another embodiment, the list can be dynamic in that additional samples can be used to update the list over time, e.g., periodically.

In one embodiment, a determination (test) for similarity does not require an exact match of the locations of the junction. One or more criteria can be used to determine if two junctions are sufficiently similar. For example, the locations of the junctions can be within a threshold distance, e.g., within one mate-pair length, which can be about 500 bp. In one implementation, the threshold distance can be independent for each edge, i.e. whether ether edge can be off by up to 500 bp. The type of junctions can also be required to match. In one implementation, the criteria can be the same or of the same type as that used to determine whether discordant mate pairs are compatible with describing a same junction (e.g., should belong to a same cluster).

In step 1330, similar junctions that occur in a sufficient number of samples are identified to obtain a list of common junctions. In one embodiment, this list can be obtained during an analysis of a new sample. In another embodiment, this list can be obtained prior to an analysis of a new sample, and the list can then be stored in any suitable storage element.

In some embodiments, a threshold is used to determine whether a sufficient number of junctions are similar enough to be classified as a group corresponding to a single junction. In various embodiments, the group can be stored by storing each of the junctions in the groups, storing a representative junction, or storing parameters that describe the group, such as an average location or a range of locations. In one embodiment, the threshold can be an absolute number (e.g., between 1 and 5) or a proportion of the number of samples used to create the list. If a proportion is used and the list is updated, some junctions could be added to a list and then removed at various update times.

In one embodiment, the junctions in the list can be restricted to interchromosomal junctions. The number inter-chromosomal events in a genome is usually small, and thus would be very unlikely and can be considered errors when in two or more samples. But, if the errors are random, most mate pairs would be inter-chromosomal (generally greater than 90%). Therefore, a mistake in mapping is most likely inter-chromosomal.

In one embodiment, steps 1320 and 1330 are not performed and every junction is added to the list. In such an embodiment, a similarity of the junctions can be addressed at a later time.

In step 1340, potential junctions are identified for a new sample. These junctions can be identified in a same or different way than the junctions identified in step 1310.

In step 1350, potential junctions for the new sample that are similar with the known junctions in the list are determined. In one embodiment, the new potential junctions can be compared to the junctions in the list. As for step 1330, an exact match is not required. For example, similar criteria can be used to determine if the junction of the new sample is similar enough to a junction on the list.

In step 1360, the potential junctions that are found on the list are marked. In an embodiment where the list includes all junctions that have been identified before, the number of similar junctions found is assessed in determining whether to filter the junction (e.g., mark). The same criteria that can be used for creating a list of similar junctions (e.g., as described above for step 1320) can be used. In another embodiment, where junctions have been combined but the number of original junctions contributing to a group is recorded, that number can be part of the marking.

Marked junctions can be discarded based on this factor or in conjunction with other results (e.g., of other filters). In one embodiment, the mark can be used in another analysis. For example, in some rearrangements, a junction can occur in two places since a total of four parts of the genome would be involved. For example, sequences of 1-2 and 3-4 could become 1-4 and 3-2. If an interchromosomal event is seen in both directions, then the junctions could be more likely to be identifying a real event. Thus, extra data can be used with the results of step 1360.

In one embodiment, such extra data and analysis of the interrelation of junctions can be used in creating the list. In another embodiment, the list can be conditional in that, if a first junction is found, it is determined whether a corresponding junction also exists in the sample genome, where the existence of the corresponding junction would signify that the junction actually is not a false positive. In another embodiment, a potential junction could be marked with a score that relates to the proportion of the samples in which a junction is typically found.

F. Narrow Clusters

Another embodiment can look at a shape of a cluster of discordant mate pairs to determine whether the cluster is likely a false positive. For example, if a cluster is very narrow, then the cluster can likely result from a mismapping around a small variation. The variation could be a small variation between the sample and the genome. Thus, arm reads at this variation point will not map properly to that part of the reference genome due to the mapping variation.

Looking at FIG. 5, cluster 550 shows data points occurring in a narrow band. As mentioned above, cluster 550 could result from a mismapping around a small variation. For example, the first arm reads with the variation might map (although with some low confidence) to another part of the reference genome. Therefore, the distance between the corresponding arm reads and the mismapped arm reads is incorrect. Since the locations of the corresponding arm reads are affected by the statistical variation in the mate-gap lengths, the result is a group of points that is narrow in one dimension. Since there is enough density in the cluster, previous clustering filters may not get rid of these occurrences. In one aspect, such mismappings might be reduced some by a realignment step. In one embodiment, discordant mate pairs that are in a narrow cluster are not likely to succeed in the junction assembly.

In some embodiments, the criteria for testing for a narrow cluster is a dimension along a single direction, e.g., horizontal or vertical on plot 500. In one embodiment, the first and last starting positions of the mappings along one dimension of a cluster must be at least a specified distance (e.g., 50 base pairs) apart from one another.

G. Absolute or As a Factor

Any of the filters mentioned herein can be absolute in nature or can be used only as a factor as part of a final determination to include a junction in a final list. In some embodiments, absolute filters can discard a discordant mate pair, cluster, or junction if certain criteria are not satisfied. For example, a cluster with a density that is too high can be discarded as a single source clone. In another embodiment, the density could still be used as a factor, e.g., if the density is close to a cutoff value, e.g., either just above or just below, then the luster may be marked instead of discarded.

In other embodiments, the results of a filtering process can be used along with the results of other filtering processes or other criteria. For example, whether a junction, cluster, or discordant mate pair satisfies a particular criterion (e.g., a filter as mentioned herein) can be used in conjunction with the results of a filtering process, where both are just factors and not determinative. In one embodiment, any of the filtering processes can provide a confidence score of how likely (or unlikely) a false positive is. These scores could then be summed, e.g., in a weighted sum where certain filters are weighted more than other. The final sum could then be compared to a threshold to determine how to classify the junction, cluster, and/or discordant mate pair.

In one embodiment, each potential junction has a column for a result of each analysis performed for the junction. These columns can then be analyzed to determine whether the junction is displayed to a user, stored in a file for likely junctions, or otherwise indicated as a likely junction. In another embodiment, different criteria could be used for different samples or junctions. For example, a researcher may want to find novel differences between a tumor and the regular genome of the person. If so, then a researcher may want to include weakly supported junctions in the normal sample. However, a sample from the tumor would likely get more stringent filters.

H. Combination of Filters

As mentioned above, any combination of filters may be used. In one embodiment, the following filters are used in the following order. Clusters are determined using discordant mate pairs. Clusters consistent with a single source clone and with too few discordant mate pairs are discarded. For the remaining clusters, a realignment is attempted for the discordant mate pairs of the cluster. Clusters surviving realignment (e.g., the discordant mate pairs could not be realigned) can be subjected to junction assembly, tests for repetitive elements, baselining, and a determination of how narrow the cluster is. The junctions undergoing these filtering processes can be marked based on the results (e.g., a pass/fail or a score), and a final determination can be made as to whether a junction is likely to exist.

### IV. Identification of Other Variations

Up until now, the identification of a junction region has mainly focused on the use of discordant mate pairs. However, other methods may be used to identify regions that may contain a junction. Any of the identification methods can be used with any of the appropriate filtering mechanisms described herein.

A. Length Distribution

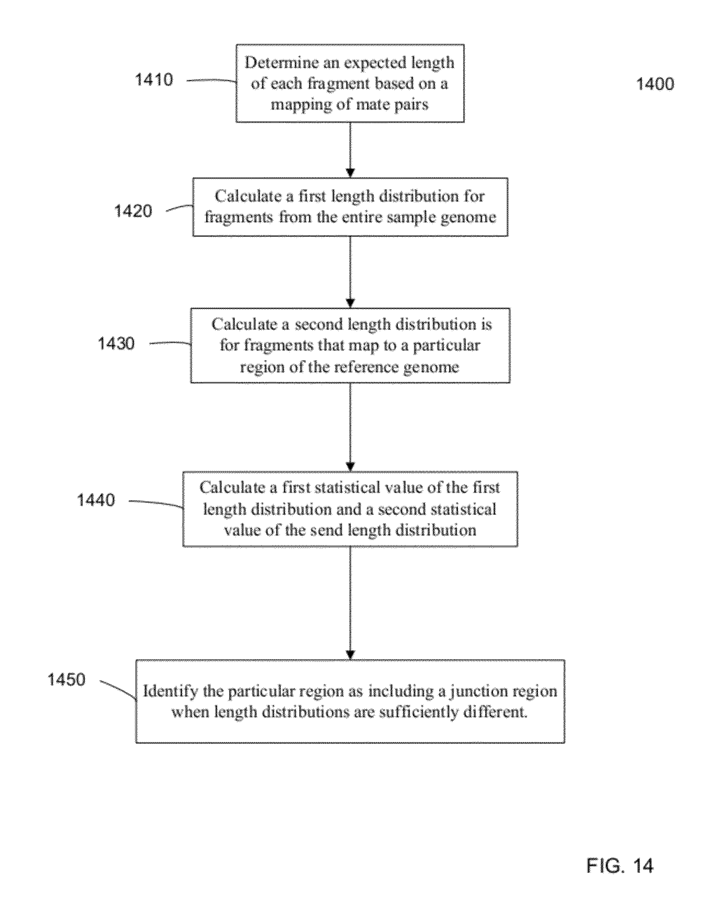

FIG. 14 is a flowchart illustrating a method 1400 for determining whether a junction exists between a sample genome and a reference genome using a distribution of fragment lengths according to embodiments of the present invention. For example, method 1400 can analyze the lengths of fragments for a particular region to identify whether a junction exists, e.g., an insertion or deletion.

In step 1410, an expected length of each fragment is determined based on the mapping of the mate pairs. In one embodiment, each fragment of a sample can be sequenced at the ends to provide a mate pair of arm reads. The arm reads can then be mapped to the reference genome. Based on the locations of the mapping, the length of the fragments can be determined. In one embodiment, only fragments with the appropriate orientation (i.e., FB) are used. In another embodiment, fragments with a length that is very short or very long may be discarded as outliers, which may have resulted, for example, from spurious biochemical events or mismappings.

In step 1420, a first length distribution is calculated for fragments from the entire sample genome. In one embodiment, the length distribution can be determined as a histogram providing the number of fragments at each length. In another embodiment, the length distribution may be a functional fit to the length data. Such histograms or functional fits would typically have a bell-shaped distribution with an average length having the most fragments. Due to statistical variation such a distribution would deviate to different degrees from the idealized distribution.

In yet another embodiment, the length distribution may simply be a disordered list of the lengths, from which a statistical value can be computed. Besides a length distribution for the present sample, a length distribution may be obtained for other samples, as the length distribution may be assumed to be similar.

In step 1430, a second length distribution is calculated for fragments that map to a particular region of the reference genome. Fragments can map to a particular region in a variety of way, as described in the following examples. In one embodiment, the particular region can be defined via predetermined start and end locations in the genome. In various implementations, a fragment can be included in a particular region if one of the arm reads is at least partially mapped to the particular region. A fragment can be included in a particular region if one of the arm reads is completely within the particular region. A fragment can be required to map entirely within the particular region. In another embodiment, a region is defined by the mate pairs that span a particular location in the genome. For example, the mate pairs that have an arm read on one side of the location and the other arm read on the opposite side of the location may be used.

In some embodiments, different particular regions can be swept through, and a different distribution determined for each region. The various regions can have the same length or different lengths, or be defined in different way (e.g., in the ways mentioned above). For the example, the method could use regions of 1000 base pairs long (or more). Once an initial region starting from the beginning of a strand is swept, the region can be advanced, for example, by a set number of base pairs (e.g., by 50 base pairs). In one embodiment, a distribution can be determined at each of many locations in the genome, e.g., every 50 base pairs.

In step 1440, a first statistical value is calculated for the first length distribution, and a second statistical value is calculated for the second length distribution. In various embodiments, a statistical value can be an average (mean) length, a median length, a length having a maximum number of fragments, ranges within a certain standard deviation of probability that a length would occur, or any other value derived from the lengths of the distribution.

In step 1450, a particular region is identified as including a junction region when the two distributions are sufficiently different. In one embodiment, a difference between the first statistical value and the second statistical value can be compared to a threshold value, e.g., to see if the threshold is exceeded. In some embodiments, the difference could be a simple subtraction of average lengths. In other embodiments, more complicated differences may be used, such as a function of a subtracted value or a subtraction of two functions of the respective values. In one implementation, the difference is taken as an absolute value and then compared to the threshold. Thus, which value is subtracted from which value may not change the exceeding of a threshold. The sign of the difference may suggest a particular type of junction however.

As an example, a large positive difference could result when an insertion has occurred in the sample genome. The insertion would cause fragments to appear longer, since some arm reads would map to the left side of the insertion, and the corresponding arm reads would map to the right side of the insertion. Similarly, a large negative difference could result from a deletion. “Positive” and “negative” are used here simply to show the opposite trends of an insertion and a deletion. Depending on the formula used for the difference calculation, either sign can suggest an insertion or deletion.

In one embodiment, the location of the particular region could be determined during a sweep of regions by identifying the specific region that provides a local maximum for the difference. In this manner, the location of the junction can be narrowed to just one region as opposed to many regions, each of which might include the junction region. Accordingly, in one embodiment, a plurality of additional length distributions and corresponding statistical values can be calculated for different regions. As mentioned above, each region can be defined by fragments that overlap with a particular part of the sample genome. The region with the maximum difference between the statistical values can be identified as the location of a potential junction, since that is the region that shows the biggest difference among a set of neighboring regions. The junction region can then be determined from the particular region with the maximum. In an embodiment where the particular regions are defined by mate pairs spanning a location, the junction region can be determined from the length distribution. For example, junction region can be centered around the location and can have a total size that can accommodate fragment lengths having a non-negligible probability of existing (e.g., greater than 0.5%). As another example, the differences for the plurality of particular regions can be plotted and the shape of this difference plot can provide an estimate for the junction region. For instance, the difference plot can have a plateau region or a region above the threshold, which can mark the size of the junction region.

In some embodiments, using the length distributions can find relatively small length-changing variations (e.g., about 100 bp). Such small variations may not be detected via discordant mate pairs since the variations are small. In one embodiment, the distribution of mate-pair lengths can be analyzed over every point in the genome. In another embodiment, junction assembly can be used to determine the sequences throughout the identified junction region.

B. Identification of Inserted Transposons

Individuals' genomes have common transposable elements at various positions, which may differ from the reference genome. A transposon is a self-replicating sequence. If there is an insertion of a transposon, then there would be many left-arm reads that map to the reference genome with the right-arm reads mapping to the transposon. These right-arm reads would not map to the reference genome due to the transposon not being at the location in the reference genome. Also, there would be right arms on the other side of the insertion with the left arms mapping to the insertion location. Accordingly, in one embodiment, novel locations of common transposable elements can be searched for by investigating areas having a concentration of arm reads that do not match the reference, but that match to a transposon sequence. Filtering and assembly methods described here can be used to improve accuracy of predicted transposon insertions.

### V. Computational Specifics for Junction Assembly

As mentioned above, junction assembly can reconstruct a likely sequence in a junction region from arm reads that do map to the junction region or may map to the junction region based on a location of a mapped arm read near the junction region. The assembly of the likely sequence from the arm reads can involve an optimization process.

In one embodiment, to set up the optimization procedure, a pool of arm reads, e.g., arm reads of DNA NanoBalls (DNB), that can contribute to assembly in the active (junction) region has already been identified. The idea consists of using this pool of DNBs to perform a de novo assembly of the sequence in the region of interest.

Previously existing assembly methods based on de Bruijn graphs apply to contiguous reads without gaps, and therefore cannot be used directly to perform de novo assembly from mated reads having variable gaps. Briefly, existing assembly methods based on de Bruijn graphs include choosing an assembly length, n_{c}<l, where l is the read length. A graph is constructed in which each vertex corresponds to a sequence of length n_{c} present in at least one of the reads. A directed edge between vertex V₁ and vertex V₂ is then created if both of the following conditions are true: 1) The sequence associated with vertex V₂ can be obtained from the sequence associated with vertex V₁ by removing its first base and adding a new base at the end. This is the definition of an edge of the de Bruijn graph. Associated with such an edge is the sequence of n_{c}+1 bases consisting of the first n_{c} bases of the sequence associated with vertex V₁ plus the last base of the sequence associated with vertex V₂; and 2) there is at least one read containing the sequence that would be associated with the directed edge.

For example, suppose n_{c}=5 is chosen and there are the following reads of length 6: CTACGA, TACGAC, ACGACT. We can then construct the following De Bruijn graph: CTACG->TACGA->ACGACG->CGACT.

The three sample reads may be associated with graph edges, in top-to-bottom order. An assembled sequence can be obtained by simply following paths in the graph. Heterozygous events and assembly uncertainties may be represented by branches in the graph. Repeats of length greater than n_{c} manifest themselves as loops—that is, the directed graph is no longer acyclic.

Such a procedure can be used with sequence arms if it is accepted that there are only individual l-base reads (e.g., 10). However, in one embodiment, the left and right arms may each include 4 contiguous reads, which in turn, each comprise three 10-base reads and one 5-base read. Therefore, the above is not acceptable as it would have the effect of neglecting the 5 bases in the 5-base read of each arm and, more important, would not use the information on the relative position of the 10-base reads implied by the presence of 10-base reads in a single arm.

In one embodiment, a de Bruijn graph procedure has been modified as follows to process variably gapped reads.

For the de Bruijn graph, the process may include selecting an assembly length n_{c} that is greater than a length l of a read, e.g., approximately 30 bases. A graph is initialized with vertices, but not edges, using the reference sequence Gₒ in the junction region. The graph is configured to comprise sequences of length nc bases associated to vertices and sequences of nc+1 bases associated to edges.

In one embodiment, an edge is allowed to be added to the graph only if we have at least a minimum number of arms that map, at least partially and without too many mismatches, to the sequence associated with that edge. In another embodiment, a local DNB index can then be used to find DNB arms that allow recursively adding additional vertices and edges to the graph. In most cases this recursive procedure is well behaved. However, in some cases the number of vertices and edges generated can diverge exponentially.

Accordingly, in one embodiment, new vertices are added to a priority queue that is ordered by vertex strength, where the vertex strength is based on a number of mapped mated reads that suggest existence of the vertex as well as a quality of their mapping to the vertex. At each step of the recursive procedure, the highest priority vertex is removed from the priority queue and tested for the ability to construct new edges to or from that vertex. The recursive procedure ends when the queue is empty, such that that no additional edges and vertices can be added to the graph, or alternatively when a certain maximum number of vertices have been created.

When finished, paths in the graph along the edges that begin and end at the first and last location in Go are enumerated. Each path provides a new seed sequence for the optimization procedure. If a total of nₚ paths are found, including the path corresponding to the reference sequence in that active interval, there are a total

$$( \frac{{n}_{P}}{P} )$$

combinations of p of the seed sequences, where p is the ploidy in the junction region.

The probability L(G) is computed for each of the combinations of seed sequences. The paths with starting sequence hypotheses having the largest probabilities L(G) (e.g., the top 3) are then used in turn as starting sequence hypotheses for the optimization procedure. In addition, the allele combination consisting of the reference for all p alleles is always also used as a seed. This limits the number of optimizations that have to be performed, which can be important in cases when the de Bruijn graph is complex and nₚ is large.

### VI. Computer System

Any of the computer systems mentioned herein may utilize any suitable number of subsystems. Examples of such subsystems are shown in FIG. 6 in computer apparatus 600. In some embodiments, a computer system includes a single computer apparatus, where the subsystems can be the components of the computer apparatus. In other embodiments, a computer system can include multiple computer apparatuses, each being a subsystem, with internal components.

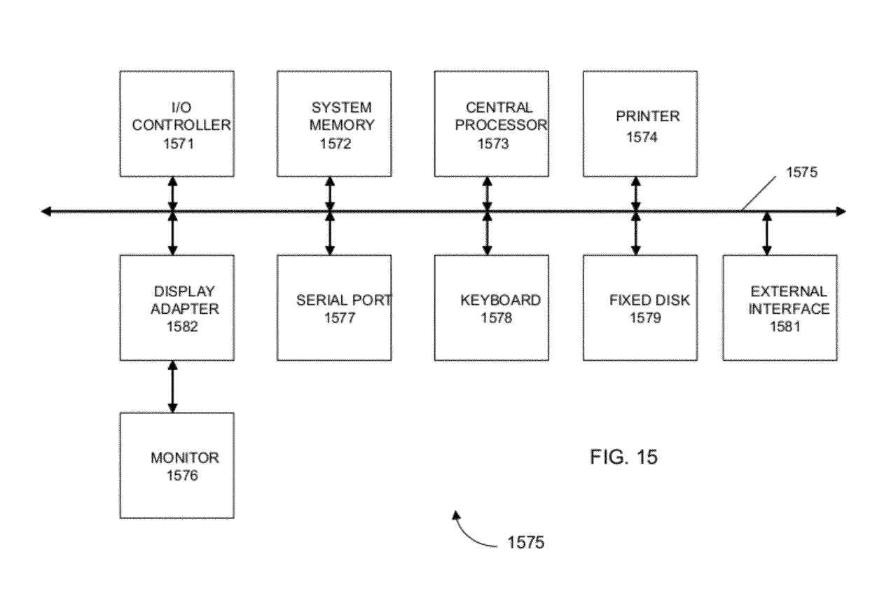

The subsystems shown in FIG. 15 are interconnected via a system bus 1575. Additional subsystems such as a printer 1574, keyboard 1578, fixed disk 1579, monitor 1576, which is coupled to display adapter 1582, and others are shown. Peripherals and input/output (I/O) devices, which couple to I/O controller 1571, can be connected to the computer system by any number of means known in the art, such as serial port 1577. For example, serial port 1577 or external interface 1581 can be used to connect computer system 1500 to a wide area network such as the Internet, a mouse input device, or a scanner. The interconnection via system bus 1575 allows the central processor 1573 to communicate with each subsystem and to control the execution of instructions from system memory 1572 or the fixed disk 1579, as well as the exchange of information between subsystems. The system memory 1572 and/or the fixed disk 1579 may embody a computer readable medium. Any of the values mentioned herein can be output from one component to another component and can be output to the user.

A computer system can include a plurality of the same components or subsystems, e.g., connected together by external interface 1581 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

It should be understood that any of the embodiments of the present invention can be implemented in the form of control logic using hardware and/or using computer software in a modular or integrated manner. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g., a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including a processor, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The above description of exemplary embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated.

A recitation of “a”, “an” or “the” is intended to mean “one or more” unless specifically indicated to the contrary.

All patents, patent applications, publications, and descriptions mentioned above are herein incorporated by reference in their entirety for all purposes. None is admitted to be prior art.

What is claimed is:

1. A method of determining whether a junction exists between a sample genome and a reference genome, the sample genome being of an organism providing a biological sample, the method comprising:
receiving results of paired-end sequencing of a plurality of fragments from the biological sample, the results including mate pairs of fragments and mappings of the mate pairs to the reference genome, wherein a mate pair includes a first arm read for a first end of a fragment and a corresponding arm read of an opposite end of the fragment;
identifying a junction region in the sample genome based on the mappings of the mate pairs to the reference genome, the junction region including:
a first edge portion including a first edge of the junction region;
a second edge portion including a second edge of the junction region, the first edge opposite the second edge; and
a potential junction between the first edge and the second edge;
identifying a first set of first arm reads, each at least partially mapping to the first edge portion or having a non-negligible probability to at least partially map to the first edge portion based on a mapped location of the respective corresponding arm read; and
comparing the sequences of the first arm reads of the first set to each other to determine whether a junction exists in the junction region.

2. The method of claim 1, wherein the respective corresponding arm reads map to the reference genome at a location proximal to the first edge portion or proximal to the second edge

3. The method of claim 1, wherein the first set includes first arm reads of concordant mate pairs.

4. The method of claim 1, wherein a junction is determined not to exist when the first arm reads of the first set are not consistent with a sequence that starts in a first region of the reference genome and that ends in a second region of the reference genome.

5. The method of claim 1, wherein a first arm read has a non-negligible probability to at least partially map to the first edge portion when the respective corresponding arm read is located within an expected length range based on a statistical distribution of fragment lengths.

6. The method of claim 1, wherein the comparison proceeds by:
identifying an initial subset of one or more first arm reads of the first set that map onto the first edge, and then
proceeding to find one or more other first arms reads of the first set that overlap the first arms of the initial subset, that include base pairs toward the potential junction; and then
comparing the other first arm reads to the reference genome to determine whether the other first arm reads include a junction.

7. The method of claim 1, further comprising:
identifying a second set of first arm reads, each at least partially mapping to the second edge portion or having a non-negligible probability to at least partially map to the second edge portion based on a mapped location of the respective corresponding arm read, wherein the respective corresponding arm reads of the first arm reads of the second set map to the reference genome at a location proximal to the second edge portion; and
comparing the sequences of the first arm reads of the second set to determine whether a junction exists in the junction region.

8. The method of claim 7, wherein at least one first arm read at least partially maps to both the first edge portion and to the second edge portion or has a non-negligible probability to at least partially map to both the first edge portion and to the second edge portion.

9. The method of claim 8, wherein the at least one first arm read is not mapped to the reference genome in the received results.

10. The method of claim 7, further comprising:
comparing the sequences of the first arm reads of the first set and the second set to determine a junction sequence of the sample genome within the junction region; and
identifying whether the junction sequence includes a junction by:
comparing the junction sequence to the reference genome to determine whether the junction sequence does not appear contiguous in the reference genome.

11. The method of claim 10, wherein a junction is determined not to exist if the junction sequence is contiguous in the reference genome.

12. The method of claim 10, wherein the first arm reads of the first set and the second set are sufficiently similar to provide a junction sequence with a probability greater than a threshold.

13. The method of claim 10, wherein the junction is where the junction sequence diverges from the reference genome.

14. The method of claim 1, wherein identifying a potential junction includes:
determining a group of discordant mate pairs, each including a first arm read mapped to a first region of the reference genome, the first region being outside of the junction region at a first side, and each including a corresponding arm read that maps to the reference genome at a location other than on the an opposite side of the junction region relative to the first side.

15. The method of claim 14, wherein determining the group of discordant mate pairs includes:
determining a plurality of discordant mate pairs based on the mapping results;
clustering the discordant mate pairs based on locations of the first arms reads and of the corresponding arm reads; and
determining the group of discordant mate pairs from the discordant mate pairs of one of the clusters.

16. The method of claim 15, wherein the plurality of discordant mate pairs includes different types of discordant mate pairs, the different types including:
mate pairs whose mate gap is larger than a threshold,
mate pairs whose order is incorrect, and
mate pairs that have a improper orientation, wherein the clustering is performed separately for each type of type of discordant mate pairs.

17. The method of claim 15, wherein determining the group of discordant mate pairs further includes:
for each cluster, determining a density of the discordant mate pairs in the cluster based on the locations of the first arm reads and the corresponding arm reads; and
identifying clusters of discordant mate pairs that have a density above a specified density value, wherein the group of discordant mate pairs is composed of the discordant mate pairs of one of the identified clusters.

18. The method of claim 1, wherein identifying a potential junction includes:
determining an expected length of each fragment based on the mapping of the mate pairs;
calculating a first length distribution for fragments from the sample genome;
calculating a second length distribution for fragments that map to a particular region of the reference genome; and
identifying the particular region as including at least part of the junction region when a difference between a first statistical value of the first length distribution and a second statistical value of the send length distribution exceeds a threshold value.

19. The method of claim 18, wherein identifying the particular region includes:
determining a plurality of particular regions having a respective difference exceeding the threshold value;
determining a first of the plurality of particular regions that has a local maximum for the difference; and
using the first particular region to define the first and second edges of the junction region.

20. The method of claim 18, wherein the difference is taken between the means of the first and second length distributions.

21. The method of claim 18, wherein a shift to smaller lengths for the second length distribution relative to the first length distribution signifies a deletion in the particular region.

22. The method of claim 18, wherein a shift to larger lengths for the second length distribution relative to the first length distribution signifies an insertion in the particular region.

23. The method of claim 18, further comprising:
calculating a plurality of additional length distribution for different regions;
calculating a plurality of additional statistical values of the additional length distributions; and
identifying a region having a maximum difference between the first statistical value and a respective statistical value as a location of a potential junction.

24. The method of claim 23, wherein each region is defined by fragments that overlap with a different part of the sample genome.

25. A computer program product comprising a tangible computer readable medium storing a plurality of instructions for controlling a processor to perform an operation for determining whether a junction exists between a sample genome and a reference genome, the sample genome being of an organism providing a biological sample, the instructions comprising the steps of the method of claim 1.

26. A method of determining whether a clinically significant junction exists between a sample genome and a reference genome, the sample genome being of an organism providing a biological sample, the method comprising:
receiving results of paired-end sequencing of a plurality of fragments from the biological sample, the results including mate pairs of fragments and mappings of the mate pairs to the reference genome, wherein a mate pair includes a first arm read for a first end of a fragment and a corresponding arm read of an opposite end of the fragment;
determining a plurality of discordant mate pairs;
determining a plurality of potential junctions based on the discordant mate pairs;
obtaining a list of junctions that have appeared in other sample genomes;
for each of the potential junctions:
determining whether the potential junction is on the list; and
determining whether or not the potential junction is a clinically significant junction based at least on whether the potential junction is on the list, wherein a potential junction that is on the list is less likely to be a clinically significant junction.

27. The method of claim 26, further comprising:
determining whether a potential junction is clinically significant based on locations of the two parts of the reference genome.

28. The method of claim 26, wherein a first potential injunction connecting a first part of the reference genome with a second part of the reference genome, the method further comprising:
for the first potential injunction, searching for repetitive elements in the first part and/or the second part of the reference genome, the repetitive elements being sequences that repeat in the reference genome;
determining whether the first potential junction is a clinically significant junction based on whether the first and second parts contain repetitive elements, wherein a potential junction is less likely to be a clinically significant junction when the first and second parts do contain repetitive elements.

29. The method of claim 28, wherein the repetitive elements that are searched are of a particular set of classes of repetitive elements.

30. The method of claim 28, further comprising:
when a repetitive element is identified for a potential junction, determining whether the identified repetitive element is similar to one or more of the arm reads of the discordant mate pairs of the potential junction,
wherein determining whether the first potential junction is a clinically significant junction is based on whether the identified repetitive element is similar to the one or more of the arm reads.

31. A method of determining whether a junction exists between a sample genome and a reference genome, the sample genome being of an organism providing a biological sample, the method comprising:
receiving results of paired-end sequencing of a plurality of fragments from the biological sample, the results including mate pairs of fragments and mappings of the mate pairs to the reference genome, wherein a mate pair includes a first arm read for a first end of a fragment and a corresponding arm read of an opposite end of the fragment;
determining a plurality of discordant mate pairs based on the mapping results;
clustering the discordant mate pairs based on locations of the first arms reads and of the corresponding arm reads;
for a plurality of the discordant mate pairs of a first cluster, attempting to perform a realignment to the reference genome of each arm of a discordant mate pair, the realignment of an arm being in a region determined from a length distribution of the fragments;
determining an amount of the plurality of discordant mate pairs of the first cluster that are realigned in a concordant manner; and
determining that a junction does not exist for the first cluster if the amount is greater than a threshold.

32. The method of claim 31, further comprising:
for each cluster, determining a density of the discordant mate pairs in the cluster based on the locations of the first arm reads and the corresponding arm reads;
discarding clusters of discordant mate pairs that have a density lower than a specified density value, thereby determining that a junction does not exist for the discarded clusters.

33. The method of claim 31, further comprising:
for each cluster, determining a largest distance between two arm reads along at least one dimension; and
discarding a cluster when the largest distance is smaller than a specified value.
